SEQUENCE LISTING (29 sequences)

SEQ ID NO: 1 - protein, 119 aa, Artificial Sequence
  Synthetic sequence
EVQLVQSGAEVKKPGESLKISCKGSGYSFTSYWIGWVRQMPGKGLEWMGIIYPGDSDTRYSPSFQGQVTISADKSISTAYLQWSSLKASDTAVYYCAKDLAPSAYFDYWGQGTLVTVSS

SEQ ID NO: 2 - protein, 120 aa, Artificial Sequence
  Synthetic sequence
EVQLVQSGAEVKKPGESLKISCKGSGYSFTNSYWIGWVRQMPGKGLEWMGIIYPGDSDTRYSPSFQGQVTISADKSISTAYLQWSSLKASDTAVYYCAKDLAPSAYFDYWGQGTLVTVSS

SEQ ID NO: 3 - protein, 120 aa, Artificial Sequence
  Synthetic sequence
EVQLVQSGAEVKKPGESLKISCKGSGYSFTNSYWIGWVRQMPGKGLEWMGMIYPGDSDTRYSPSFQGQVTISADKSISTAYLQWSSLKASDTAVYYCAKDLAPSAYFDYWGQGTLVTVSS

SEQ ID NO: 4 - protein, 120 aa, Artificial Sequence
  Synthetic sequence
ELQLVQSGAEVKKPGESLKISCKGSGYSFTNSYWIGWVRQMPGKGLEWMGMIYPGDSDTRYSPSFQGQVTISADKSISTAYLQWSSLKASDTAVYYCAKDLAPSAYFDYWGQGTLVTVSS

SEQ ID NO: 5 - protein, 120 aa, Artificial Sequence
  Synthetic sequence
ELQLVQSGAEVKKPGESLKISCKGSGYSFTNSYWIGWVRQMPGKGLEWMGMIYPGDSDTRYSPSFQGQVTISNDKSISTAYLQWNSLKASDTAVYYCAKDLAPSAYFDYWGQGTLVTVSS

SEQ ID NO: 6 - protein, 120 aa, Artificial Sequence
  Synthetic sequence
ELQLVQSGAEVKKPGESLKISCKGSGYSFTNSYWIGWVRQMPGKGLEWMGMIYPGDSDTRYSPSFQGQVTISNDKSISTAYLQWASLKASDTAVYYCAKDLAPSAYYDYWGQGTLVTVSS

SEQ ID NO: 7 - protein, 120 aa, Artificial Sequence
  Synthetic sequence
ELQLVQSGAEVKKPGESLKISCKGSGYSFSNSYWIGWVRQMPGKGLEWMGMIYPGDSDTRYSPSFQGQVTISNDKSISTAYLQWASLKASDTAVYYCAKDLAPSAYFNYWGQGTLVTVSS

SEQ ID NO: 8 - protein, 120 aa, Artificial Sequence
  Synthetic sequence
ELQLVQSGAEVKKPGESLKISCKGSGYSFTNSYWIGWVRQMPGKGLEWMGMIYPGDSDTRYSPSFQGQVTISNDKSISTAYLQWNSLKASDTAVYYCAKDLAPSAYFNYWGQGTLVTVSS

SEQ ID NO: 9 - protein, 120 aa, Artificial Sequence
  Synthetic sequence
ELQLVQSGAEVKKPGESLKISCKGSGYSFTNSYWIGWVRQMPGKGLEWMGMIYPGDSDTRYSPSFQGQVTISNDKSISTAYLQWASLKASDTAVYYCAKDLAPSAYYNYWGQGTLVTVSS

SEQ ID NO: 10 - protein, 120 aa, Artificial Sequence
  Synthetic sequence
ELQLVQSGAEVKKPGESLKISCKGSGYSFTNSYWIGWVRQMPGKGLEWMGMIYPGDSDTRYSPSFQGQVTISNDKSISTAYLQWNSLKASDTAVYYCAKDLAPSAYYNYWGQGTLVTVSS

SEQ ID NO: 11 - protein, 120 aa, Artificial Sequence
  Synthetic sequence
ELQLVQSGAEVKKPGESLKISCKGSGYSFSNSYWIGWVRQMPGKGLEWMGMIYPGDSDTRYSPSFQGQVTISNDKSISTAYLQWASLKASDTAVYYCAKDLAPSAYYNYWGQGTLVTVSS

SEQ ID NO: 12 - protein, 120 aa, Artificial Sequence
  Synthetic sequence
ELQLVQSGAEVKKPGESLKISCKGSGYSFSNSYWIGWVRQMPGKGLEWMGMIYPGDSDTRYSPSFQGQVTISNDKSISTAYLQWNSLKASDTAVYYCAKDLAPSAYYNYWGQGTLVTVSS

SEQ ID NO: 13 - protein, 123 aa, Artificial Sequence
  Synthetic sequence
QVQLVQSGAEVKKPGASVKVSCKASGYTFTGYYMHWVRQAPGQGLEWMGRINPNSGGTNYAQKFQGRVTSTRDTSISTAYMELSRLRSDDTAVYYCAKAKWFGDVNREVFDYWGQGTLVTVSS

SEQ ID NO: 14 - protein, 123 aa, Artificial Sequence
  Synthetic sequence
QVQLVQSGAEVKKPGASVKVSCKASGYTFIGYYMHWVRQAPGQGLEWMGRINPNSGGTNYAQKFQGRVTSTRDTSISTAYMELSRLRSDDTAVYYCAKAKWFGDVNREVFDYWGQGTLVTVSS

SEQ ID NO: 15 - protein, 116 aa, Artificial Sequence
  Synthetic sequence
WLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPYTFGQGTRLEIKR

SEQ ID NO: 16 - protein, 116 aa, Artificial Sequence
  Synthetic sequence
WLPDTTGEIVLTQSPGTLSLSPGERATLSCRASQSVRSSYLAWYQQKPGQAPRLLIYGASSRATGIPDRFSGSGSGTDFTLTISRLEPEDFAVYYCQQYGSSPYTFGQGTRLEIKR

SEQ ID NO: 17 - protein, 119 aa, Artificial Sequence
  Synthetic sequence
WLPDTTGEIVLTQSPGTLSLSPGERATLSCRASQSVSSSYLAYLAWYQQKPGQAPRLLIYGASSRATGIPDRFSGSGSGTDFTLTISRLEPEDFAVYYCQQYGSSPYTFGQGTRLEIKR

SEQ ID NO: 18 - protein, 119 aa, Artificial Sequence
  Synthetic sequence
WLPDTTGEIVLTQSPGTLSLSPGERATLSCRASQSVRSSYLAYLAWYQQKPGQAPRLLIYGASSRATGIPDRFSGSGSGTDFTLTISRLEPEDFAVYYCQQYGSSPYTFGQGTRLEIKR

SEQ ID NO: 19 - protein, 119 aa, Artificial Sequence
  Synthetic sequence
WLPDTTGEIVLTQSPGTLSLSPGERATLSCRASQSVRSSYLAYLAWYQQKPGQAPRLLIYGASSRATGIPDRFSGSGSGTDFTLTISRLEPEDFAVYYCQQYGSLPYTFGQGTRLEIKR

SEQ ID NO: 20 - protein, 119 aa, Artificial Sequence
  Synthetic sequence
WLPDTTGEIVLTQSPGTLSLSPGERATLSCRASQSVRSSYLAYLAWYQQKPGQAPRLLIYDASSRATGIPDRFSGSGSGTDFTLTISRLEPEDFAVYYCQQYGSLPYTFGQGTRLEIKR

SEQ ID NO: 21 - protein, 119 aa, Artificial Sequence
  Synthetic sequence
WLPDTTGEIVLTQSPGTLSLSPGERATLSCRASQSVRSSYLAYLAWYQQKPGQAPRLLIYGASSRATGIPDRFSGSGSGTDFTLTISRLEPEDFAVYYCQQYGRLPYTFGQGTRLEIKR

SEQ ID NO: 22 - protein, 119 aa, Artificial Sequence
  Synthetic sequence
WLPDTTGEIVLTQSPGTLSLSPGERATLSCRASQSVRSSYLAYLAWYQQKPGQAPRLLIYDASSRATGIPDRFSGSGSGTDFTLTISRLEPEDFAVYYCQQYGRLPYTFGQGTRLEIKR

SEQ ID NO: 23 - protein, 109 aa, Artificial Sequence
  Synthetic sequence
DIQMTQSPSSLSASVGDRVTITCRASQSISSYLNWYQQKPGKAPKLLIYAASSLQSGVPSRFSGSGSGTDFTLTISSLQPEDFAVYYCMQGTHWPYTFGGGTKVEIKRT

SEQ ID NO: 24 - protein, 109 aa, Artificial Sequence
  Synthetic sequence
DIQMTQSPSSLSASVGDRVTITCRTSQSISSYLNWYQQKPGKAPKLLIYAASSLQSGVPSRFSGSGSGTDFTLTISSLQPEDFAVYYCMQGTHWPYTFGGGTKVEIKRT

SEQ ID NO: 25 - protein, 109 aa, Artificial Sequence
  Synthetic sequence
DIQMTQSPSSLSASVGDRVTITCRTSQSISSYLNWYQQKPGKAPKLLIYAASSLQSGVPSRFSGSESGTDFTLTISSLQPEDFAVYYCMQGTHWPYTFGGGTKVEIKRT

SEQ ID NO: 26 - protein, 120 aa, Artificial Sequence
  Synthetic sequence
EVQLVQSGAEVKKPGESLKISCKGSGYSFTNSYWIGWVRQMPGKGLEWMGMIYPGDSDTRYSPSFQGQVTISSDKSISTAYLQWNSLKASDTAVYYCAKDLAPSAYYNYWGQGTLVTVSS

SEQ ID NO: 27 - protein, 120 aa, Artificial Sequence
  Synthetic sequence
EVQLVQSGAEVKKPGESLKISCKGSGYSFTNSYWIGWVRQMPGKGLEWMGMIYPGDSDTRYSPSFQGQVTISSDKSISTAYLQWNSLKASDTAVYYCAKDLAPSAYYDYWGQGTLVTVSS

SEQ ID NO: 28 - protein, 120 aa, Artificial Sequence; identical to SEQ ID NO: 26
  Synthetic sequence

SEQ ID NO: 29 - protein, 120 aa, Artificial Sequence
  Synthetic sequence
EVQLVQSGAEVKKPGESLKISCKGSGYSFTNSYWIGWVRQMPGKGLEWMGMIYPGDSDTRYSPSFQGQVTISSDKSISTAYLQWASLKASDTAVYYCAKDLTPSAYYDYWGQGTLVTVSS

(19) United States
(12) Patent Application Publication
(10) Pub. No.: US 2017/0158762 A1
(43) Pub. Date: Jun. 8, 2017

(54) ANTIBODIES DIRECTED AGAINST DISCOIDIN DOMAIN RECEPTOR FAMILY, MEMBER 1 (DDR1)

(71) Applicant: AnaptysBio, Inc., San Diego, CA (US)

(72) Inventors: Helen Toni Jun, San Diego, CA (US); Robert A. Horlick, San Diego, CA (US); David J. King, Encinitas, CA (US)

(21) Appl. No.: 15/115,574

(22) PCT Filed: Jan. 27, 2015
(86) PCT No.: PCT/US2015/013027

§ 371 Date: Jul. 29, 2016

Related U.S. Application Data
(60) Provisional application No. 61/933,601, filed on Jan. 30, 2014.

(51) Int. Cl. C07K 16/28

(52) U.S. Cl. CPC C07K 16/28; C07K 2317/56; C07K 2317/92

(57) ABSTRACT

The invention relates to an isolated immunoglobulin heavy chain polypeptide and an isolated immunoglobulin light chain polypeptide that bind to Discoidin Domain Receptor Family, Member 1 (DDR1). The invention provides a DDR1-binding agent that comprises the aforementioned immunoglobulin heavy chain polypeptide and immunoglobulin light chain polypeptide. The invention also provides related vectors, compositions, and methods of using the DDR1-binding agent to treat a DDR1-mediated disease.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 32,976 Byte ASCII (Text) file named “719554_ST25.TXT,” created on Jan. 22, 2015.

### BACKGROUND OF THE INVENTION

Discoidin domain receptors (DDRs) DDR1 and DDR2 were initially characterized as receptor tyrosine kinases (RTKs) based on the presence of a catalytic kinase domain (KD) (see, e.g., Johnson et al., Proc. Natl. Acad. Sci. U.S.A., 90: 5677-5681 (1993); Di Marco et al., J. Biol. Chem., 268: 24290-24295 (1993); Zerlin et al., Oncogene, 8: 2731-2739 (1993); Perez et al., Oncogene, 12: 1469-1477 (1996); Laval et al., Cell Growth Differ., 5: 1173-1183 (1994); Sanchez et al., Proc. Natl. Acad. Sci. U.S.A., 91: 1819-1823 (1994); and Alves et al., Oncogene, 10: 609-618 (1995)). Subsequently, collagens were identified as ligands for DDRs (Vogel et al., Mol. Cell, 1: 13-23 (1997)). Upon collagen binding, DDRs undergo tyrosine autophosphorylation, which elicits genetic and cellular programs that regulate a variety of cell-collagen interactions. The biochemical and cellular mechanisms by which DDRs mediate their multiple biological effects are not well defined. The DDR1 subfamily is composed of five membrane-anchored isoforms, and the DDR2 subfamily is represented by a single protein (see, e.g., Fu et al., J. Biol. Chem., 288(11): 7430-7437 (2013)). The five DDR1 isoforms are generated by alternative splicing. DDR1a, DDR1b, and DDR1c are full-length functional receptors, and DDR1d and DDR1e are truncated or kinase-inactive receptors (see, e.g., Valiathan et al., Cancer Metastasis Rev., 31, 295-321 (2012); and Alves et al., FASEB J., 15: 1321-1323 (2001)). DDR1b and DDR1c contain an additional 37 residues within the intracellular juxtamembrane (IJXM) region. With the exception of the two secreted DDR1 isoforms, all DDRs are single-pass type I transmembrane glycoproteins that are characterized by the presence of six distinct protein domains: a discoidin (DS) domain, a DS-like domain, an extracellular juxtamembrane (EJXM) region, a transmembrane (TM) segment, a long IJXM region, and an intracellular kinase domain (KD). The presence of the N-terminal DS and DS-like domains is the defining feature of the DDR RTK subfamily. The DS domain exhibits high homology to a protein moiety originally identified in proteins from Dictyostelium discoideum.

Both DDR1 and DDR2 bind to and are activated by fibrillar collagens I-III and V. Basement membrane collagen IV activates DDR1 but not DDR2 (see, e.g., Vogel et al., Mol. Cell, 1: 13-23 (1997); and Leitinger, B., J. Biol. Chem., 278: 16761-16769 (2003)), whereas non-fibrillar collagen X primarily activates DDR2 (see, e.g., Leitinger and Kwan, Matrix Biol., 25: 355-364 (2006)). The primary binding site for both DDR1 and DDR2 in collagens II and III contains the GVMGFO motif (where O is hydroxyproline), which is also conserved in the α1(I) and α1(V) chains (see, e.g., Gu et al., PLoS ONE, 6: e15640 (2011); and Giudici et al., J. Biol. Chem., 283: 19551-19560 (2008)). However, none of the six α(IV) chains contain the GVMGFO motif (see, e.g., Parkin et al., Hum. Mutat., 32: 127-143 (2011)), suggesting the possibility that other sites in nonfibrillar collagens are involved in DDR1 binding. DDRs bind to the native triple helix of collagens and not to individual collagen α-chains or denatured collagens (Vogel et al., supra; and Leitinger, supra).

Recent studies have shown that DDR1 is abnormally expressed in human tumors, including lung, esophageal, breast, ovary, and pediatric brain cancers, suggesting that DDR1 plays a role in the development of human cancers (see, e.g., Kim et al., J. Biol. Chem., 286(20): 17672-17681 (2011); Johnson et al., Proc. Natl. Acad. Sci. U.S.A., 90: 10891 (1993); Alves et al., Oncogene, 10: 609-618 (1995); Heinzelmann-Schwarz, et al., Clin. Cancer Res., 10: 4427-4436 (2004); Weiner et al., Neurosurgery, 47: 1400-1409 (2000); Nemoto et al., Pathobiology, 65: 195-203 (1997); Quan et al., Int. J. Mol. Sci., 12(2): 971-982 (2011); Ford et al., Br. J. Cancer, 96: 808-814 (2007); Rikova et al., Cell, 131(6): 1190-1203 (2007); Yang et al., Oncol. Rep., 24(2): 311-319 (2010); Barker et al., Oncogene, 10(3): 569-575 (1995) and Chiaretti et al., Clin. Cancer Res., 11: 7209-7219 (2005)). Elevated expression of DDR1 also has been observed in a number of fast growing invasive tumors, suggesting that DDR1 may be involved in the proliferation and stroma invasion of tumors (see, e.g., Park et al., Oncol. Rep., 18: 1435-1441 (2007)). The mechanism by which this receptor may contribute to oncogenesis is not yet known; however, evidence suggests that it may act as a critical regulator of cell proliferation, adhesion, migration, and subsequent tumor metastasis (Ford et al., supra).

DDR1-deficient mice have been shown to be protected against bleomycin-induced pulmonary fibrosis (see, e.g., Avivi-Green et al., Am. J. Respir. Crit. Care Med., 174(4): 420-427 (2006)). In particular, collagen deposition, tenascin-C upregulation, myofibroblast expansion, and apoptosis were reduced in DDR1 knock-out mice following bleomycin challenge, as were inflammatory responses (e.g., macrophage and lymphocyte infiltration, IL-6 expression, and macrophage chemoattractant (MCP-1) expression). These results suggest that the expression of DDR1 on inflammatory cells is a prerequisite for the development of lung inflammation and fibrosis.

DDR1-deficient mice also have been shown to be protected against renal fibrosis associated with hypertension and obstructive nephropathy (see, e.g., Guerrot et al., Am. J. Pathol., 179(1): 83-91 (2011)), suggesting that DDR1 plays an important role in the pathogenesis of renal disease by enhancing the inflammatory response.

Therefore, there is a need for inhibitors of DDR1 (e.g., antibodies) that bind DDR1 with high affinity and effectively neutralize DDR1 activity. The invention provides a DDR1 binding agent that binds to and inhibits DDR1.

### BRIEF SUMMARY OF THE INVENTION

The invention provides an isolated immunoglobulin heavy chain polypeptide which comprises an amino acid sequence of SEQ ID NO: 1, wherein optionally one or more of residues 2, 30, 31, 51, 78, 85, 102, 107, and 108 of SEQ ID NO: 1 is replaced with a different amino acid residue.

The invention provides an isolated immunoglobulin light chain polypeptide which comprises an amino acid sequence of SEQ ID NO: 13, wherein optionally residue 30 of SEQ ID NO: 13 is replaced with a different amino acid residue.

The invention provides an isolated immunoglobulin light chain polypeptide which comprises an amino acid sequence of SEQ ID NO: 15, wherein optionally: (a) one or more of residues 37, 58, 101, and 102 of SEQ ID NO: 15 is replaced with a different amino acid residue, and/or (b) an amino acid sequence comprising YLA is inserted into SEQ ID NO: 15 after residue 42.

The invention provides an isolated immunoglobulin light chain polypeptide which comprises an amino acid sequence of SEQ ID NO: 23, wherein optionally reside 25 and/or residue 66 of SEQ ID NO: 23 is/are replaced with a different amino acid residue.

In addition, the invention provides isolated or purified nucleic acid sequences encoding the foregoing immunoglobulin polypeptides, vectors comprising such nucleic acid sequences, isolated DDR1-binding agents comprising the foregoing immunoglobulin polypeptides, nucleic acid sequences encoding such DDR1-binding agents, vectors comprising such nucleic acid sequences, isolated cells comprising such vectors, compositions comprising such DDR1-binding agents or such vectors with a pharmaceutically acceptable carrier, and methods of treating a DDR1-mediated disorder in mammals by administering effective amounts of such compositions to mammals.

### BRIEF DESCRIPTION OF THE SEVERAL VIEWS OF THE DRAWING(S)

FIG. 1 is a graph illustrating experimental data depicting DDR1 epitope binning of the anti-DDR1 antibodies described in Example 1 by 3E3 monoclonal antibody competition assay. Cells expressing surface displayed antibodies representing three of the lead strategies were incubated with DDR1 bio:NA Dyl650 (4:1) complexes in the presence of 6-fold excess 3E3 antibody or control huIgG and analyzed by FACS array. An irrelevant antigen control was also included as a measure of background binding. Mean fluorescence intensity (MFI) was graphed on the Y axis to quantify binding.

FIG. 2 is a graph illustrating experimental data depicting SHM mutations identified in the anti-DDR1 antibodies by next generation sequencing (NGS). Mutations (squares) were identified by NGS as enriching based on their representation in sequence results by Illumina sequencing. 30,000-100,000 reads were performed and analyzed using a proprietary software algorithm. Improving mutations had either increased antigen binding above the reference line (upper panel) or decreased KD below the reference line (lower panel). Mutations were then incorporated into the best HC or LC context available at the time and compared to the parental antibody (dots).

FIG. 3 is a graph illustrating experimental data depicting inhibition of collagen-mediated phosphorylation of DDR1 in the DiscoveRx U2OS assay described in Example 3. Cells were plated at 20K cells per well and allowed to incubate for 30 minutes before addition of antibody. Cells were stimulated 90 minutes later with 25 μg/ml collagen II and allowed to incubate overnight. Cells were lysed and GALACTON-STAR™ chemiluminescent substrate (Life Technologies, Carlsbad, Calif.) was added along with EMERALD II™ stabilizer and read on the ENVISION™ plate reader (Life Technologies, Carlsbad, Calif.). Curves were fit using GraphPad Prism software (GraphPad, San Diego, Calif.) and normalized to % inhibition to allow for comparison among plates.

FIG. 4 is a graph illustrating experimental data depicting results of an ELISA assay to detect binding of antibodies APE05570 and APE05571 to human DDR1. Antibodies were incubated in normal mouse serum (NMS) for indicated time at 37 C. Antibodies were then analyzed for ability to bind to DDR1 by ELISA.

### DETAILED DESCRIPTION OF THE INVENTION

The invention provides an isolated immunoglobulin heavy chain polypeptide and an isolated immunoglobulin light chain polypeptide, or a fragment (e.g., antigen-binding fragment) thereof. The term “immunoglobulin” or “antibody,” as used herein, refers to a protein that is found in blood or other bodily fluids of vertebrates, which is used by the immune system to identify and neutralize foreign objects, such as bacteria and viruses. The polypeptide is “isolated” in that it is removed from its natural environment. In a preferred embodiment, an immunoglobulin or antibody is a protein that comprises at least one complementarity determining region (CDR). The CDRs form the “hypervariable region” of an antibody, which is responsible for antigen binding (discussed further below). A whole immunoglobulin typically consists of four polypeptides: two identical copies of a heavy (H) chain polypeptide and two identical copies of a light (L) chain polypeptide. Each of the heavy chains contains one N-terminal variable (V_{H}) region and three C-terminal constant (C_{H}1, C_{H}2, and C_{H}3) regions, and each light chain contains one N-terminal variable (V_{L}) region and one C-terminal constant (C_{L}) region. The light chains of antibodies can be assigned to one of two distinct types, either kappa (κ) or lambda (λ) based upon the amino acid sequences of their constant domains. In a typical immunoglobulin, each light chain is linked to a heavy chain by disulphide bonds, and the two heavy chains are linked to each other by disulphide bonds. The light chain variable region is aligned with the variable region of the heavy chain, and the light chain constant region is aligned with the first constant region of the heavy chain. The remaining constant regions of the heavy chains are aligned with each other.

The variable regions of each pair of light and heavy chains form the antigen binding site of an antibody. The V_{H} and V_{L} regions have the same general structure, with each region comprising four framework (FW or FR) regions. The term “framework region,” as used herein, refers to the relatively conserved amino acid sequences within the variable region which are located between the hypervariable or complementary determining regions (CDRs). There are four framework regions in each variable domain, which are designated FR1, FR2, FR3, and FR4. The framework regions form the β sheets that provide the structural framework of the variable region (see, e.g., C. A. Janeway et al. (eds.), Immunobiology, 5th Ed., Garland Publishing, New York, N.Y. (2001)).

The framework regions are connected by three complementarity determining regions (CDRs). As discussed above, the three CDRs, known as CDR1, CDR2, and CDR3, form the “hypervariable region” of an antibody, which is responsible for antigen binding. The CDRs form loops connecting, and in some cases comprising part of, the beta-sheet structure formed by the framework regions. While the constant regions of the light and heavy chains are not directly involved in binding of the antibody to an antigen, the constant regions can influence the orientation of the variable regions. The constant regions also exhibit various effector functions, such as participation in antibody-dependent complement-mediated lysis or antibody-dependent cellular toxicity via interactions with effector molecules and cells.

The isolated immunoglobulin heavy chain polypeptide and the isolated immunoglobulin light chain polypeptide of the invention desirably bind to DDR1. As discussed above, Discoidin Domain Receptor Family, Member 1 (DDR1) is a receptor tyrosine kinase activated by various types of collagens that is involved in cell attachment, migration, survival, and proliferation (L'Hôte et al., FASEB J., 16: 234-236 (2002)). DDR1 kinase is distinct from other members of the large receptor tyrosine kinase due to a homology domain to discoidin, which is a lectin first described during the cell aggregation process of the slime mold Dictyostelium discoideum (see, e.g., Vogel W., FASEB J., 13: S77-82 (1999); and Schlessinger, J., Cell, 91: 869-872 (1997)).

Five isoforms of DDR1 are generated by alternative splicing. DDR1a, DDR1b, and DDR1c are full-length functional receptors, and DDR1d and DDR1e are truncated or kinase-inactive receptors (see, e.g., Valiathan et al., supra, and Alves et al., FASEB J., 15: 1321-1323 (2001)). DDR1-mediated signaling is initiated by collagen binding, after which DDR1 undergoes tyrosine autophosphorylation, which elicits genetic and cellular programs that regulate a variety of cell-collagen interactions.

DDRs have been found widely expressed in human and mouse tissues, such as, for example, the brain, keratinocytes, the epithelial layer of the colonic mucosa, the distal tubules of the kidney, lung epithelium, and thyroid follicles (see, e.g., Johnson et al., Proc. Natl. Acad. Sci. USA, 70: 2554-2557 (1973); Perez et al., Oncogene, 9: 211-219 (1994); Sanchez et al., Proc. Natl. Acad. Sci. USA, 91: 1819-1823 (1994); Di Marco et al., J. Biol. Chem., 268: 24290-24295 (1993); Alves et al., Oncogene, 10: 609-618 (1995); and Laval et al., Cell Growth Diff., 5: 1173-1183 (1994)). In the pancreas, DDR1 expression is restricted to the islets of Langerhans (Alves et al., Oncogene, 10: 609-618 (1995)). During mouse development, DDR1 can be used as an early marker for the formation of neuroectodermal cells (see, e.g., Zerlin, et al., Oncogene, 8: 2731-2739 (1993)). As discussed above, DDR1 overexpression occurs in a variety of human cancers, particularly breast, ovarian, and non-small cell lung carcinomas (NSCLCs).

The inventive isolated immunoglobulin heavy chain polypeptide and the inventive isolated immunoglobulin light chain polypeptide each bind to DDR1 and preferably do not bind to DDR2, or alternatively, bind to DDR2 with low affinity. In other words, the inventive isolated immunoglobulin heavy and light chain polypeptides specifically bind to DDR1. By “low affinity,” is meant that the inventive isolated immunoglobulin heavy chain polypeptide and/or the inventive isolated immunoglobulin light chain polypeptide binds to DDR2 with an affinity that is at least 100-fold weaker (e.g., 100-fold, 200-fold, 300-fold, 400-fold, or 500-fold weaker) than the affinity for DDR1.

Antibodies which bind to DDR1, and components thereof, are known in the art (see, e.g., International Patent Application Publications WO 2010/019702, WO 2013/034933, and WO 2013/027802). Anti-DDR1 antibodies also are commercially available from sources such as, for example, Santa Cruz Biotechnology (Dallas, Tex.).

One example of an immunoglobulin heavy chain polypeptide that binds to the DDR1 protein comprises the amino acid sequence of SEQ ID NO: 1. In one embodiment of the invention, the isolated immunoglobulin heavy chain polypeptide comprises an amino acid sequence of SEQ ID NO: 1, wherein optionally one or more of residues 2, 30, 31, 51, 78, 85, 102, 107, and 108 of SEQ ID NO: 1 is replaced with a different amino acid residue. In one embodiment of the invention, the isolated immunoglobulin heavy chain polypeptide comprises, consists of, or consists essentially of an amino acid sequence of SEQ ID NO: 1, wherein optionally one or more of residues 2, 30, 31, 51, 78, 85, 102, 107, and 108 of SEQ ID NO: 1 is replaced with a different amino acid residue. When the inventive immunoglobulin heavy chain polypeptide consists essentially of an amino acid sequence of SEQ ID NO: 1 and optional amino acid replacements, additional components can be included in the polypeptide that do not materially affect the polypeptide (e.g., protein moieties such as biotin that facilitate purification or isolation). When the inventive immunoglobulin heavy chain polypeptide consists of an amino acid sequence SEQ ID NO: 1 and optional amino acid replacements, the polypeptide does not comprise any additional components (i.e., components that are not endogenous to the inventive immunoglobulin heavy chain polypeptide).

Each of amino acid residues 2, 30, 31, 51, 78, 85, 102, 107, and 108 of SEQ ID NO: 1 can be replaced with any suitable amino acid residue that can be the same or different in each position. For example, the amino acid residue of a first position can be replaced with a first different amino acid residue, and the amino acid residue of a second position can be replaced with a second different amino acid residue, wherein the first and second different amino acid residues are the same or different. An amino acid “replacement” or “substitution” refers to the replacement of one amino acid at a given position or residue by another amino acid at the same position or residue within a polypeptide sequence.

Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or Ile), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gln), lysine (K or Lys), and arginine (R or Arg).

Aliphatic amino acids may be sub-divided into four sub-groups. The “large aliphatic non-polar sub-group” consists of valine, leucine, and isoleucine. The “aliphatic slightly-polar sub-group” consists of methionine, serine, threonine, and cysteine. The “aliphatic polar/charged sub-group” consists of glutamic acid, aspartic acid, asparagine, glutamine, lysine, and arginine. The “small-residue sub-group” consists of glycine and alanine. The group of charged/polar amino acids may be sub-divided into three sub-groups: the “positively-charged sub-group” consisting of lysine and arginine, the “negatively-charged sub-group” consisting of glutamic acid and aspartic acid, and the “polar sub-group” consisting of asparagine and glutamine.

Aromatic amino acids may be sub-divided into two sub-groups: the “nitrogen ring sub-group” consisting of histidine and tryptophan and the “phenyl sub-group” consisting of phenylalanine and tyrosine.

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra).

Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH₂ can be maintained.

“Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

In one embodiment, the isolated immunoglobulin heavy chain polypeptide comprises the amino acid sequence of SEQ ID NO: 1, except that (a) residue 2 of SEQ ID NO: 1 is replaced with a leucine (L) residue, (b) residue 30 of SEQ ID NO: 1 is replaced with a serine (S) residue, (c) residue 31 of SEQ ID NO: 1 is replaced with an asparagine (N) residue, (d) residue 51 of SEQ ID NO: 1 is replaced with a methionine (M) residue, (e) residue 78 of SEQ ID NO: 1 is replaced with an asparagine (N) residue, (f) residue 85 of SEQ ID NO: 1 is replaced with an asparagine (N) residue (g) residue 102 of SEQ ID NO: 1 is replaced with a threonine (T) residue, (h) residue 107 of SEQ ID NO: 1 is replaced with a tyrosine (Y) residue, (i) residue 108 of SEQ ID NO: 1 is replaced with an asparagine (N) residue, or (j) any combination of two or more of the foregoing replacements.

Exemplary immunoglobulin heavy chain polypeptides as described above can comprise any one of the following amino acid sequences: SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 26, SEQ ID NO: 27, SEQ ID NO: 28, or SEQ ID NO: 29.

In addition, one or more amino acids can be inserted into the aforementioned immunoglobulin heavy chain polypeptides. Any number of any suitable amino acids can be inserted into the amino acid sequence of the immunoglobulin heavy chain polypeptide. In this respect, at least one amino acid (e.g., 2 or more, 5 or more, or 10 or more amino acids), but not more than 20 amino acids (e.g., 18 or less, 15 or less, or 12 or less amino acids), can be inserted into the amino acid sequence of the immunoglobulin heavy chain polypeptide. Preferably, 1-10 amino acids (e.g., 1, 2, 3, 4, 5, 6, 7, 8, 9, or 10 amino acids) are inserted into the amino acid sequence of the immunoglobulin heavy chain polypeptide. In this respect, the amino acid(s) can be inserted into any one of the aforementioned immunoglobulin heavy chain polypeptides in any suitable location. Preferably, the amino acid(s) are inserted into a CDR (e.g., CDR1, CDR2, or CDR3) of the immunoglobulin heavy chain polypeptide.

The invention provides an isolated immunoglobulin heavy chain polypeptide which comprises an amino acid sequence that is at least 90% identical (e.g., at least 91%, at least 92%, at least 93%, at least 94%, at least 95%, at least 96%, at least 97%, at least 98%, at least 99%, or 100% identical) to any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 26, SEQ ID NO: 27, SEQ ID NO: 28, or SEQ ID NO: 29. Nucleic acid or amino acid sequence “identity,” as described herein, can be determined by comparing a nucleic acid or amino acid sequence of interest to a reference nucleic acid or amino acid sequence. The percent identity is the number of nucleotides or amino acid residues that are the same (i.e., that are identical) as between the sequence of interest and the reference sequence divided by the length of the longest sequence (i.e., the length of either the sequence of interest or the reference sequence, whichever is longer). A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and PASTA programs (e.g., FASTA3x, FASTM, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probablistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

The invention provides an immunoglobulin light chain polypeptide that comprises an amino acid sequence of SEQ ID NO: 13, wherein optionally residue 30 of SEQ ID NO: 13 is replaced with a different amino acid residue. In one embodiment of the invention, the isolated immunoglobulin light chain polypeptide comprises, consists of, or consists essentially of an amino acid sequence of SEQ ID NO: 13, wherein optionally residue 30 of SEQ ID NO: 13 is replaced with a different amino acid residue. When the inventive immunoglobulin light chain polypeptide consists essentially of an amino acid sequence of SEQ ID NO: 13 and optional amino acid replacements, additional components can be included in the polypeptide that do not materially affect the polypeptide (e.g., protein moieties such as biotin that facilitate purification or isolation). When the inventive immunoglobulin light chain polypeptide consists of an amino acid sequence of SEQ ID NO: 13 and optional amino acid replacements, the polypeptide does not comprise any additional components (i.e., components that are not endogenous to the inventive immunoglobulin light chain polypeptide).

Amino acid residue 30 of SEQ ID NO: 13 can be replaced with any suitable amino acid residue. In one embodiment, the isolated immunoglobulin light chain polypeptide comprises the amino acid sequence of SEQ ID NO: 13, except that residue 30 of SEQ ID NO: 13 is replaced with an isoleucine (I) residue. An exemplary immunoglobulin light chain polypeptide can comprise SEQ ID NO: 14.

The invention also provides an isolated immunoglobulin light chain polypeptide which comprises an amino acid sequence of SEQ ID NO: 15, wherein optionally: (a) one or more of residues 37, 58, 101, and 102 of SEQ ID NO: 15 is replaced with a different amino acid residue, and/or (b) an amino acid sequence comprising YLA is inserted into SEQ ID NO: 15 after residue 42. In one embodiment of the invention, the isolated immunoglobulin light chain polypeptide comprises, consists of, or consists essentially of SEQ ID NO: 15, wherein optionally: (a) one or more of residues 37, 58, 101, and 102 of SEQ ID NO: 15 is replaced with a different amino acid residue, and/or (b) an amino acid sequence comprising YLA is inserted into SEQ ID NO: 15 after residue 42. When the inventive immunoglobulin light chain polypeptide consists essentially of an amino acid sequence of SEQ ID NO: 15 and optional amino acid replacements and/or insertions, additional components can be included in the polypeptide that do not materially affect the polypeptide (e.g., protein moieties such as biotin that facilitate purification or isolation). When the inventive immunoglobulin light chain polypeptide consists of an amino acid sequence of SEQ ID NO: 15 and optional amino acid replacements and/or insertions, the polypeptide does not comprise any additional components (i.e., components that are not endogenous to the inventive immunoglobulin light chain polypeptide).

For example, the isolated immunoglobulin light chain polypeptide can comprise SEQ ID NO: 15, except that either (a) one or more of residues 37, 58, 101, and 102 of SEQ ID NO: 15 is replaced with a different amino acid residue, or (b) an amino acid sequence comprising YLA is inserted into SEQ ID NO: 15 after residue 42. Alternatively, the isolated immunoglobulin light chain polypeptide can comprise SEQ ID NO: 15, except that both (a) one or more of residues 37, 58, 101, and 102 of SEQ ID NO: 15 is replaced with a different amino acid residue, and (b) an amino acid sequence comprising YLA is inserted into SEQ ID NO: 15 after residue 42. Each of amino acid residues 37, 58, 101, and 102 of SEQ ID NO: 15 can be replaced with any suitable amino acid residue that can be the same or different in each position. For example, the amino acid residue of a first position can be replaced with a first different amino acid residue, and the amino acid residue of a second position can be replaced with a second different amino acid residue, wherein the first and second different amino acid residues are the same or different.

In one embodiment, the isolated immunoglobulin light chain polypeptide comprises SEQ ID NO: 15, except that (a) residue 37 of SEQ ID NO: 15 is replaced with an arginine (R) residue, (b) residue 58 of SEQ ID NO: 15 is replaced with an aspartic acid (D) residue, (c) residue 101 of SEQ ID NO: 15 is replaced with an arginine (R) residue, (d) residue 102 of SEQ ID NO: 15 is replaced with a leucine (L) residue, or any combination of two or more of the foregoing replacements. An exemplary immunoglobulin light chain polypeptide can comprise SEQ ID NO: 16.

In addition, one or more amino acids can be inserted into the aforementioned immunoglobulin light chain polypeptide. Any number of any suitable amino acids can be inserted into the amino acid sequence of the immunoglobulin light chain polypeptide. In this respect, at least one amino acid (e.g., 2 or more, 5 or more, or 10 or more amino acids), but not more than 20 amino acids (e.g., 18 or less, 15 or less, or 12 or less amino acids), can be inserted into the amino acid sequence of the immunoglobulin light chain polypeptide. Preferably, 1-10 amino acids (e.g., 2, 3, 4, 5, 6, 7, 8, or 9 amino acids) are inserted in to the amino acid sequence of the immunoglobulin light chain polypeptide. In this respect, the amino acids can be inserted into SEQ ID NO: 15 in any suitable location. Preferably, the amino acid(s) are inserted into a CDR (e.g., CDR1, CDR2, or CDR3) of SEQ ID NO: 15. In one embodiment, the amino acid(s) are inserted into CDR1 of SEQ ID NO: 15. For example, an amino acid sequence comprising YLA can be inserted into SEQ ID NO: 15 after residue 42. As discussed above, the inventive immunoglobulin light chain polypeptide comprising SEQ ID NO: 15 can include an amino acid insertion alone, or in combination with one or more amino acid replacements and/or deletions described herein. Exemplary immunoglobulin light chain polypeptides as described above can comprise SEQ ID NO: 17, SEQ ID NO: 18, SEQ ID NO: 19, SEQ ID NO: 20, SEQ ID NO: 21, or SEQ ID NO: 22.

The invention also provides an isolated immunoglobulin light chain polypeptide which comprises an amino acid sequence of SEQ ID NO: 23, wherein optionally reside 25 and/or residue 66 of SEQ ID NO: 23 is/are replaced with a different amino acid residue. In one embodiment of the invention, the isolated immunoglobulin light chain polypeptide comprises, consists of, or consists essentially of an amino acid sequence of SEQ ID NO: 23, wherein optionally reside 25 and/or residue 66 of SEQ ID NO: 23 is/are replaced with a different amino acid residue. When the inventive immunoglobulin light chain polypeptide consists essentially of an amino acid sequence of SEQ ID NO: 23 and optional amino acid replacements, additional components can be included in the polypeptide that do not materially affect the polypeptide (e.g., protein moieties such as biotin that facilitate purification or isolation). When the inventive immunoglobulin light chain polypeptide consists of an amino acid sequence SEQ ID NO: 23 and optional amino acid replacements, the polypeptide does not comprise any additional components (i.e., components that are not endogenous to the inventive immunoglobulin light chain polypeptide).

Each of amino acid residues 25 and/or 66 of SEQ ID NO: 23 can be replaced with any suitable amino acid residue that can be the same or different in each position. For example, the amino acid residue of a first position can be replaced with a first different amino acid residue, and the amino acid residue of a second position can be replaced with a second different amino acid residue, wherein the first and second different amino acid residues are the same or different. In one embodiment, the isolated immunoglobulin light chain polypeptide comprises the amino acid sequence of SEQ ID NO: 23, except that residue 25 of SEQ ID NO: 23 is replaced with a threonine (T) residue and/or residue 66 of SEQ ID NO: 23 is replaced with a glutamic acid (E) residue. Exemplary immunoglobulin light chain polypeptides can comprise SEQ ID NO: 24 or SEQ ID NO: 25.

The invention provides an isolated immunoglobulin light chain polypeptide which comprises an amino acid sequence that is at least 90% identical (e.g., at least 91%, at least 92%, at least 93%, at least 94%, at least 95%, at least 96%, at least 97%, at least 98%, at least 99%, or 100% identical) to any one of SEQ ID NOs: 13-25. Nucleic acid or amino acid sequence “identity” can be determined using the methods described herein.

The inventive isolated immunoglobulin heavy chain polypeptide and light chain polypeptides are not limited to polypeptides comprising the specific amino acid sequences described herein. Indeed, the immunoglobulin heavy chain polypeptide or light chain polypeptide can be any immunoglobulin heavy chain polypeptide or light chain polypeptide that competes with the inventive immunoglobulin heavy chain polypeptide or light chain polypeptide for binding to DDR1. In this respect, for example, the immunoglobulin heavy chain polypeptide or light chain polypeptide can be any heavy chain polypeptide or light chain polypeptide that binds to the same epitope of DDR1 recognized by the heavy or light chain polypeptides described herein, respectively. Antibody competition can be assayed using routine peptide competition assays which utilize ELISA, Western blot, or immunohistochemistry methods (see, e.g., U.S. Pat. Nos. 4,828,981 and 8,568,992; and Braitbard et al., Proteome Sci., 4: 12 (2006)).

The inventive isolated immunoglobulin heavy chain polypeptide and light chain polypeptides are not limited to polypeptides comprising the specific amino acid sequences described herein. Indeed, the immunoglobulin heavy chain polypeptide or light chain polypeptide can be any heavy chain polypeptide or light chain polypeptide that competes with the inventive immunoglobulin heavy chain polypeptide or light chain polypeptide for binding to DDR1. In this respect, for example, the immunoglobulin heavy chain polypeptide or light chain polypeptide can be any heavy chain polypeptide or light chain polypeptide that binds to the same epitope of DDR1 recognized by the heavy and light chain polypeptides described herein. The invention also provides an isolated or purified epitope of DDR1. Antibody competition can be assayed using routine peptide competition assays which utilize ELISA, Western blot, or immunohistochemistry methods (see, e.g., U.S. Pat. Nos. 4,828,981 and 8,568,992; and Braitbard et al., Proteome Sci., 4: 12 (2006)).

The invention provides an isolated Discoidin Domain Receptor Family, Member 1 (DDR1)-binding agent comprising, consisting essentially of, or consisting of one or more of the inventive isolated amino acid sequences described herein. By “DDR1-binding agent” is meant a molecule, preferably a proteinaceous molecule, that binds specifically to DDR1. Preferably, the DDR1-binding agent is an antibody or a fragment (e.g., immunogenic fragment) thereof. The isolated DDR1-binding agent of the invention comprises, consists essentially of, or consists of the inventive isolated immunoglobulin heavy chain polypeptide and/or the inventive isolated immunoglobulin light chain polypeptide. In one embodiment, the isolated DDR1-binding agent comprises, consists essentially of, or consists of either the inventive immunoglobulin heavy chain polypeptide or the inventive immunoglobulin light chain polypeptide. In another embodiment, the isolated DDR1-binding agent comprises, consists essentially of, or consists of both the inventive immunoglobulin heavy chain polypeptide and the inventive immunoglobulin light chain polypeptide.

Any amino acid residue of the inventive immunoglobulin heavy chain polypeptide and/or the inventive immunoglobulin light chain polypeptide can be replaced, in any combination, with a different amino acid residue, or can be deleted or inserted, so long as the biological activity of the DDR1-binding agent is enhanced or improved as a result of the amino acid replacements, insertions, and/or deletions. The “biological activity” of a DDR1-binding agent refers to, for example, binding affinity for a particular DDR1 epitope, neutralization or inhibition of DDR1 binding to its receptor(s), neutralization or inhibition of DDR1 activity in vivo (e.g., IC₅₀), pharmacokinetics, and cross-reactivity (e.g., with non-human homologs or orthologs of the DDR1 protein, or with other proteins or tissues). Other biological properties or characteristics of an antigen-binding agent recognized in the art include, for example, avidity, selectivity, solubility, folding, immunotoxicity, expression, and formulation. The aforementioned properties or characteristics can be observed, measured, and/or assessed using standard techniques including, but not limited to, ELISA, competitive ELISA, surface plasmon resonance analysis (BIACORE™), or KINEXA™, in vitro or in vivo neutralization assays, receptor-ligand binding assays, cytokine or growth factor production and/or secretion assays, and signal transduction and immunohistochemistry assays.

The terms “inhibit” or “neutralize,” as used herein with respect to the activity of a DDR1-binding agent, refer to the ability to substantially antagonize, prohibit, prevent, restrain, slow, disrupt, alter, eliminate, stop, or reverse the progression or severity of, for example, the biological activity of DDR1, or a disease or condition associated with DDR1. The isolated DDR1-binding agent of the invention preferably inhibits or neutralizes the activity of DDR1 by at least about 20%, about 30%, about 40%, about 50%, about 60%, about 70%, about 80%, about 90%, about 95%, about 100%, or a range defined by any two of the foregoing values.

The isolated DDR1-binding agent of the invention can be a whole antibody, as described herein, or an antibody fragment. The terms “fragment of an antibody,” “antibody fragment,” and “functional fragment of an antibody” are used interchangeably herein to mean one or more fragments of an antibody that retain the ability to specifically bind to an antigen (see, generally, Holliger et al., Nat. Biotech., 23(9): 1126-1129 (2005)). The isolated DDR1 binding agent can contain any DDR1-binding antibody fragment. The antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the V_{L}, V_{H}, C_{L}, and CH₁ domains, (ii) a F(ab′)₂ fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)₂ fragment using mild reducing conditions, (v) a disulfide-stabilized Fv fragment (dsFv), and (vi) a domain antibody (dAb), which is an antibody single variable region domain (VH or VL) polypeptide that specifically binds antigen.

In embodiments where the isolated DDR1-binding agent comprises a fragment of the immunoglobulin heavy chain or light chain polypeptide, the fragment can be of any size so long as the fragment binds to, and preferably inhibits the activity of, DDR1. In this respect, a fragment of the immunoglobulin heavy chain polypeptide desirably comprises between about 5 and 18 (e.g., about 5, 6, 7, 8, 9, 10, 11, 12, 13, 14, 15, 16, 17, 18, or a range defined by any two of the foregoing values) amino acids. Similarly, a fragment of the immunoglobulin light chain polypeptide desirably comprises between about 5 and 18 (e.g., about 5, 6, 7, 8, 9, 10, 11, 12, 13, 14, 15, 16, 17, 18, or a range defined by any two of the foregoing values) amino acids.

When the DDR1-binding agent is an antibody or antibody fragment, the antibody or antibody fragment desirably comprises a heavy chain constant region (F_{c}) of any suitable class. Preferably, the antibody or antibody fragment comprises a heavy chain constant region that is based upon wild-type IgG1, IgG2, or IgG4 antibodies, or variants thereof.

The DDR1-binding agent also can be a single chain antibody fragment. Examples of single chain antibody fragments include, but are not limited to, (i) a single chain Fv (scFv), which is a monovalent molecule consisting of the two domains of the Fv fragment (i.e., V_{L} and V_{H}) joined by a synthetic linker which enables the two domains to be synthesized as a single polypeptide chain (see, e.g., Bird et al., Science, 242: 423-426 (1988); Huston et al., Proc. Natl. Acad. Sci. USA, 85: 5879-5883 (1988); and Osbourn et al., Nat. Biotechnol., 16: 778 (1998)) and (ii) a diabody, which is a dimer of polypeptide chains, wherein each polypeptide chain comprises a V_{H} connected to a V_{L} by a peptide linker that is too short to allow pairing between the V_{H} and V_{L} on the same polypeptide chain, thereby driving the pairing between the complementary domains on different V_{H}-V_{L} polypeptide chains to generate a dimeric molecule having two functional antigen binding sites. Antibody fragments are known in the art and are described in more detail in, e.g., U.S. Patent Application Publication 2009/0093024 A1.

The isolated DDR1-binding agent also can be an intrabody or fragment thereof. An intrabody is an antibody which is expressed and which functions intracellularly. Intrabodies typically lack disulfide bonds and are capable of modulating the expression or activity of target genes through their specific binding activity. Intrabodies include single domain fragments such as isolated V_{H} and V_{L} domains and scFvs. An intrabody can include sub-cellular trafficking signals attached to the N or C terminus of the intrabody to allow expression at high concentrations in the sub-cellular compartments where a target protein is located. Upon interaction with a target gene, an intrabody modulates target protein function and/or achieves phenotypic/functional knockout by mechanisms such as accelerating target protein degradation and sequestering the target protein in a non-physiological sub-cellular compartment. Other mechanisms of intrabody-mediated gene inactivation can depend on the epitope to which the intrabody is directed, such as binding to the catalytic site on a target protein or to epitopes that are involved in protein-protein, protein-DNA, or protein-RNA interactions.

The isolated DDR1-binding agent also can be an antibody conjugate. In this respect, the isolated DDR1-binding agent can be a conjugate of (1) an antibody, an alternative scaffold, or fragments thereof, and (2) a protein or non-protein moiety comprising the DDR1-binding agent. For example, the DDR1-binding agent can be all or part of an antibody conjugated to a peptide, a fluorescent molecule, or a chemotherapeutic agent.

The isolated DDR1-binding agent can be, or can be obtained from, a human antibody, a non-human antibody, or a chimeric antibody. By “chimeric” is meant an antibody or fragment thereof comprising both human and non-human regions. Preferably, the isolated DDR1-binding agent is a humanized antibody. A “humanized” antibody is a monoclonal antibody comprising a human antibody scaffold and at least one CDR obtained or derived from a non-human antibody. Non-human antibodies include antibodies isolated from any non-human animal, such as, for example, a rodent (e.g., a mouse or rat). A humanized antibody can comprise, one, two, or three CDRs obtained or derived from a non-human antibody. In one embodiment of the invention, CDRH3 of the inventive DDR1-binding agent is obtained or derived from a mouse monoclonal antibody, while the remaining variable regions and constant region of the inventive DDR1-binding agent are obtained or derived from a human monoclonal antibody.

A human antibody, a non-human antibody, a chimeric antibody, or a humanized antibody can be obtained by any means, including via in vitro sources (e.g., a hybridoma or a cell line producing an antibody recombinantly) and in vivo sources (e.g., rodents). Methods for generating antibodies are known in the art and are described in, for example, Köhler and Milstein, Eur. J. Immunol., 5: 511-519 (1976); Harlow and Lane (eds.), Antibodies: A Laboratory Manual, CSH Press (1988); and Janeway et al. (eds.), Immunobiology, 5th Ed., Garland Publishing, New York, N.Y. (2001)). In certain embodiments, a human antibody or a chimeric antibody can be generated using a transgenic animal (e.g., a mouse) wherein one or more endogenous immunoglobulin genes are replaced with one or more human immunoglobulin genes. Examples of transgenic mice wherein endogenous antibody genes are effectively replaced with human antibody genes include, but are not limited to, the Medarex HUMAB-MOUSE™, the Kirin TC MOUSE™, and the Kyowa Kirin KM-MOUSE™ (see, e.g., Lonberg, Nat. Biotechnol., 23(9): 1117-25 (2005), and Lonberg, Handb. Exp. Pharmacol., 181: 69-97 (2008)). A humanized antibody can be generated using any suitable method known in the art (see, e.g., An, Z. (ed.), Therapeutic Monoclonal Antibodies: From Bench to Clinic, John Wiley & Sons, Inc., Hoboken, N.J. (2009)), including, e.g., grafting of non-human CDRs onto a human antibody scaffold (see, e.g., Kashmiri et al., Methods, 36(1): 25-34 (2005); and Hou et al., J. Biochem., 144(1): 115-120 (2008)). In one embodiment, a humanized antibody can be produced using the methods described in, e.g., U.S. Patent Application Publication 2011/0287485 A1.

In one embodiment, a CDR (e.g., CDR1, CDR2, or CDR3) or a variable region of the immunoglobulin heavy chain polypeptide and/or the immunoglobulin light chain polypeptide described herein can be transplanted (i.e., grafted) into another molecule, such as an antibody or non-antibody polypeptide, using either protein chemistry or recombinant DNA technology. In this regard, the invention provides an isolated DDR1-binding agent comprising at least one CDR of an immunoglobulin heavy chain and/or light chain polypeptide as described herein. The isolated DDR1-binding agent can comprise one, two, or three CDRs of an immunoglobulin heavy chain and/or light chain variable region as described herein. In this regard, the CDR1 of the immunoglobulin heavy chain polypeptides described herein is located between amino acid residues 26 and 36, inclusive, of SEQ ID NOs: 1-12 and SEQ ID NOs 26-29. The CDR2 of the immunoglobulin heavy chain polypeptides described herein is located between amino acid residues 51 and 60, inclusive, of SEQ ID NOs: 1-12 and SEQ ID NOs 26-29. The CDR3 of the immunoglobulin heavy chain polypeptides described herein is located between amino acid residues 100 and 109, inclusive, of any one of SEQ ID NOs: 1-12 and SEQ ID NOs 26-29.

The CDR1 of the immunoglobulin light chain polypeptides described herein is located between amino acid residues 26 and 35, inclusive, of SEQ ID NO: 13 and SEQ ID NO: 14, between amino acid residues 31 and 42, inclusive, of SEQ ID NOs: 15-22, and between amino acid residues 24 and 34, inclusive, of SEQ ID NOs: 23-25. The CDR2 of the immunoglobulin light chain polypeptides described herein is located between amino acid residues 50 and 59, inclusive, of SEQ ID NO: 13 and SEQ ID NO: 14, between amino acid residues 58 and 64, inclusive, of SEQ ID NOs: 15-22, and between amino acid residues 50 and 56, inclusive, of SEQ ID NOs: 23-25. The CDR3 of the immunoglobulin light chain polypeptides described herein is located between amino acid residues 99 and 112, inclusive, of SEQ ID NO: 13 and SEQ ID NO: 14, between amino acid residues 97 and 105, inclusive, of SEQ ID NOs: 15-22, and between amino acid residues 89 and 97, inclusive, of SEQ ID NOs: 23-25.

In a preferred embodiment, the DDR1-binding agent binds an epitope of DDR1 which blocks the binding of DDR1 to collagens (e.g., fibrillar collagens I-III and V) and inhibits DDR1 mediated signaling. The invention also provides an isolated or purified epitope of DDR1 which blocks the binding of DDR1 to collagens in an indirect or allosteric manner.

The invention also provides one or more isolated or purified nucleic acid sequences that encode the inventive immunoglobulin heavy chain polypeptide, the inventive immunoglobulin light chain polypeptide, and the inventive DDR1-binding agent.

The term “nucleic acid sequence” is intended to encompass a polymer of DNA or RNA, i.e., a polynucleotide, which can be single-stranded or double-stranded and which can contain non-natural or altered nucleotides. The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to, methylated and/or capped polynucleotides. Nucleic acids are typically linked via phosphate bonds to form nucleic acid sequences or polynucleotides, though many other linkages are known in the art (e.g., phosphorothioates, boranophosphates, and the like).

The invention further provides a vector comprising one or more nucleic acid sequences encoding the inventive immunoglobulin heavy chain polypeptide, the inventive immunoglobulin light chain polypeptide, and/or the inventive DDR1-binding agent. The vector can be, for example, a plasmid, episome, cosmid, viral vector (e.g., retroviral or adenoviral), or phage. Suitable vectors and methods of vector preparation are well known in the art (see, e.g., Sambrook et al., Molecular Cloning, a Laboratory Manual, 3rd edition, Cold Spring Harbor Press, Cold Spring Harbor, N.Y. (2001), and Ausubel et al., Current Protocols in Molecular Biology, Greene Publishing Associates and John Wiley & Sons, New York, N.Y. (1994)).

In addition to the nucleic acid sequence encoding the inventive immunoglobulin heavy polypeptide, the inventive immunoglobulin light chain polypeptide, and/or the inventive DDR1-binding agent, the vector preferably comprises expression control sequences, such as promoters, enhancers, polyadenylation signals, transcription terminators, internal ribosome entry sites (IRES), and the like, that provide for the expression of the coding sequence in a host cell. Exemplary expression control sequences are known in the art and described in, for example, Goeddel, Gene Expression Technology: Methods in Enzymology, Vol. 185, Academic Press, San Diego, Calif. (1990).

A large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, the RSV promoter. Inducible promoters include, for example, the Tet system (U.S. Pat. Nos. 5,464,758 and 5,814,618), the Ecdysone inducible system (No et al., Proc. Natl. Acad. Sci., 93: 3346-3351 (1996)), the T-REX™ system (Invitrogen, Carlsbad, Calif.), LACSWITCH™ system (Stratagene, San Diego, Calif.), and the Cre-ERT tamoxifen inducible recombinase system (Indra et al., Nuc. Acid. Res., 27: 4324-4327 (1999); Nuc. Acid. Res., 28: e99 (2000); U.S. Pat. No. 7,112,715; and Kramer & Fussenegger, Methods Mol. Biol., 308: 123-144 (2005)).

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences.

The vector also can comprise a “selectable marker gene.” The term “selectable marker gene,” as used herein, refers to a nucleic acid sequence that allow cells expressing the nucleic acid sequence to be specifically selected for or against, in the presence of a corresponding selective agent. Suitable selectable marker genes are known in the art and described in, e.g., International Patent Application Publications WO 1992/008796 and WO 1994/028143; Wigler et al., Proc. Natl. Acad. Sci. USA, 77: 3567-3570 (1980); O'Hare et al., Proc. Natl. Acad. Sci. USA, 78: 1527-1531 (1981); Mulligan & Berg, Proc. Natl. Acad. Sci. USA, 78: 2072-2076 (1981); Colberre-Garapin et al., J. Mol. Biol., 150: 1-14 (1981); Santerre et al., Gene, 30: 147-156 (1984); Kent et al., Science, 237: 901-903 (1987); Wigler et al., Cell, 11: 223-232 (1977); Szybalska & Szybalski, Proc. Natl. Acad. Sci. USA, 48: 2026-2034 (1962); Lowy et al., Cell, 22: 817-823 (1980); and U.S. Pat. Nos. 5,122,464 and 5,770,359.

In some embodiments, the vector is an “episomal expression vector” or “episome,” which is able to replicate in a host cell, and persists as an extrachromosomal segment of DNA within the host cell in the presence of appropriate selective pressure (see, e.g., Conese et al., Gene Therapy, 11: 1735-1742 (2004)). Representative commercially available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, pREP7, and pcDNA3.1 from Invitrogen (Carlsbad, Calif.) and pBK-CMV from Stratagene (La Jolla, Calif.) represent non-limiting examples of an episomal vector that uses T-antigen and the SV40 origin of replication in lieu of EBNA1 and oriP.

Other suitable vectors include integrating expression vectors, which may randomly integrate into the host cell's DNA, or may include a recombination site to enable the specific recombination between the expression vector and the host cell's chromosome. Such integrating expression vectors may utilize the endogenous expression control sequences of the host cell's chromosomes to effect expression of the desired protein. Examples of vectors that integrate in a site specific manner include, for example, components of the flp-in system from Invitrogen (Carlsbad, Calif.) (e.g., pcDNA™5/FRT), or the cre-lox system, such as can be found in the pExchange-6 Core Vectors from Stratagene (La Jolla, Calif.). Examples of vectors that randomly integrate into host cell chromosomes include, for example, pcDNA3.1 (when introduced in the absence of T-antigen) from Life Technologies (Carlsbad, Calif.), UCOE from Millipore (Billerica, Mass.), and pCI or pFN10A (ACT) FLEXI™ from Promega (Madison, Wis.).

Viral vectors also can be used. Representative commercially available viral expression vectors include, but are not limited to, the adenovirus-based Per.C6 system available from Crucell, Inc. (Leiden, The Netherlands), the lentiviral-based pLP1 from Invitrogen (Carlsbad, Calif.), and the retroviral vectors pFB-ERV plus pCFB-EGSH from Stratagene (La Jolla, Calif.).

Nucleic acid sequences encoding the inventive amino acid sequences can be provided to a cell on the same vector (i.e., in cis). A unidirectional promoter can be used to control expression of each nucleic acid sequence. In another embodiment, a combination of bidirectional and unidirectional promoters can be used to control expression of multiple nucleic acid sequences. Nucleic acid sequences encoding the inventive amino acid sequences alternatively can be provided to the population of cells on separate vectors (i.e., in trans). Each of the nucleic acid sequences in each of the separate vectors can comprise the same or different expression control sequences. The separate vectors can be provided to cells simultaneously or sequentially.

The vector(s) comprising the nucleic acid(s) encoding the inventive amino acid sequences can be introduced into a host cell that is capable of expressing the polypeptides encoded thereby, including any suitable prokaryotic or eukaryotic cell. As such, the invention provides an isolated cell comprising the inventive vector. Preferred host cells are those that can be easily and reliably grown, have reasonably fast growth rates, have well characterized expression systems, and can be transformed or transfected easily and efficiently.

Examples of suitable prokaryotic cells include, but are not limited to, cells from the genera Bacillus (such as Bacillus subtilis and Bacillus brevis), Escherichia (such as E. coli), Pseudomonas, Streptomyces, Salmonella, and Erwinia. Particularly useful prokaryotic cells include the various strains of Escherichia coli (e.g., K12, HB101 (ATCC No. 33694), DH5a, DH10, MC1061 (ATCC No. 53338), and CC102).

Preferably, the vector is introduced into a eukaryotic cell. Suitable eukaryotic cells are known in the art and include, for example, yeast cells, insect cells, and mammalian cells. Examples of suitable yeast cells include those from the genera Kluyveromyces, Pichia, Rhino-sporidium, Saccharomyces, and Schizosaccharomyces. Preferred yeast cells include, for example, Saccharomyces cerivisae and Pichia pastoris.

Suitable insect cells are described in, for example, Kitts et al., Biotechniques, 14: 810-817 (1993); Lucklow, Curr. Opin. Biotechnol., 4: 564-572 (1993); and Lucklow et al., J. Virol., 67: 4566-4579 (1993). Preferred insect cells include Sf-9 and HI5 (Invitrogen, Carlsbad, Calif.).

Preferably, mammalian cells are utilized in the invention. A number of suitable mammalian host cells are known in the art, and many are available from the American Type Culture Collection (ATCC, Manassas, Va.). Examples of suitable mammalian cells include, but are not limited to, Chinese hamster ovary cells (CHO) (ATCC No. CCL61), CHO DHFR-cells (Urlaub et al., Proc. Natl. Acad. Sci. USA, 97: 4216-4220 (1980)), human embryonic kidney (HEK) 293 or 293T cells (ATCC No. CRL1573), and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70). Further exemplary mammalian host cells include primate cell lines and rodent cell lines, including transformed cell lines. Normal diploid cells, cell strains derived from in vitro culture of primary tissue, as well as primary explants, are also suitable. Other suitable mammalian cell lines include, but are not limited to, mouse neuroblastoma N2A cells, HeLa, mouse L-929 cells, and BHK or HaK hamster cell lines, all of which are available from the ATCC. Methods for selecting suitable mammalian host cells and methods for transformation, culture, amplification, screening, and purification of cells are known in the art.

Most preferably, the mammalian cell is a human cell. For example, the mammalian cell can be a human lymphoid or lymphoid derived cell line, such as a cell line of pre-B lymphocyte origin. Examples of human lymphoid cells lines include, without limitation, RAMOS (CRL-1596), Daudi (CCL-213), EB-3 (CCL-85), DT40 (CRL-2111), 18-81 (Jack et al., Proc. Natl. Acad. Sci. USA, 85: 1581-1585 (1988)), Raji cells (CCL-86), and derivatives thereof.

A nucleic acid sequence encoding the inventive amino acid sequence may be introduced into a cell by “transfection,” “transformation,” or “transduction.” “Transfection,” “transformation,” or “transduction,” as used herein, refer to the introduction of one or more exogenous polynucleotides into a host cell by using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)). Phage or viral vectors can be introduced into host cells, after growth of infectious particles in suitable packaging cells, many of which are commercially available.

The invention provides a composition comprising an effective amount of the inventive immunoglobulin heavy chain polypeptide, the inventive immunoglobulin light chain polypeptide, the inventive DDR1-binding agent, the inventive nucleic acid sequence encoding any of the foregoing, or the inventive vector comprising the inventive nucleic acid sequence. Preferably, the composition is a pharmaceutically acceptable (e.g., physiologically acceptable) composition, which comprises a carrier, preferably a pharmaceutically acceptable (e.g., physiologically acceptable) carrier, and the inventive amino acid sequences, antigen-binding agent, or vector. Any suitable carrier can be used within the context of the invention, and such carriers are well known in the art. The choice of carrier will be determined, in part, by the particular site to which the composition may be administered and the particular method used to administer the composition. The composition optionally can be sterile. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, Pa. (2001).

The invention further provides a method of treating a DDR1-mediated disorder in a mammal. The method comprises administering the aforementioned composition to a mammal having a DDR1-mediated disorder, whereupon the DDR1-mediated disorder is treated in the mammal. The term “DDR1-mediated disorder,” as used herein, refers to any disease or disorder in which the improper expression (e.g., overexpression) or increased activity of DDR1 causes or contributes to the pathological effects of the disease, or a decrease in DDR1 levels or activity has a therapeutic benefit in mammals, preferably humans. In one embodiment, the DDR1-mediated disorder can be cancer, such as, for example, breast cancer, ovarian cancer, lung cancer (e.g., non-small cell lung carcinoma (NSCLC)), melanoma, renal cell carcinoma, brain cancer, esophageal cancer, bladder cancer, cervical cancer, colon cancer, liver cancer, thyroid cancer, stomach cancer, salivary gland cancer, prostate cancer, or pancreatic cancer. In another embodiment, the DDR-mediated disease can be fibrosis. Fibrosis can occur in a variety of different tissue or organ systems, typically as a result of inflammation or damage that can be caused by other diseases or disorders. The inventive DDR1-binding agent can be used to treat fibrosis that occurs in any tissue of a mammal (e.g., a human), including, for example, the lung (e.g., pulmonary fibrosis or cystic fibrosis), liver (e.g., cirrhosis), heart (e.g., atrial fibrosis or endomyocardial fibrosis), kidney (e.g., renal fibrosis), and skin (e.g., scleroderma).

As used herein, the terms “treatment,” “treating,” and the like refer to obtaining a desired pharmacologic and/or physiologic effect. Preferably, the effect is therapeutic, i.e., the effect partially or completely cures a disease and/or adverse symptom attributable to the disease. To this end, the inventive method comprises administering a “therapeutically effective amount” of the DDR1-binding agent. A “therapeutically effective amount” refers to an amount effective, at dosages and for periods of time necessary, to achieve a desired therapeutic result. The therapeutically effective amount may vary according to factors such as the disease state, age, sex, and weight of the individual, and the ability of the DDR1-binding agent to elicit a desired response in the individual. For example, a therapeutically effective amount of a DDR1-binding agent of the invention is an amount which decreases DDR1 bioactivity in a human.

Alternatively, the pharmacologic and/or physiologic effect may be prophylactic, i.e., the effect completely or partially prevents a disease or symptom thereof. In this respect, the inventive method comprises administering a “prophylactically effective amount” of the DDR1-binding agent. A “prophylactically effective amount” refers to an amount effective, at dosages and for periods of time necessary, to achieve a desired prophylactic result (e.g., prevention of disease onset).

A typical dose can be, for example, in the range of 1 pg/kg to 20 mg/kg of animal or human body weight; however, doses below or above this exemplary range are within the scope of the invention. The daily parenteral dose can be about 0.00001 μg/kg to about 20 mg/kg of total body weight (e.g., about 0.001 μg/kg, about 0.1 μg/kg, about 1 μg/kg, about 5 μg/kg, about 10 μg/kg, about 100 μg/kg, about 500 μg/kg, about 1 mg/kg, about 5 mg/kg, about 10 mg/kg, or a range defined by any two of the foregoing values), preferably from about 0.1 μg/kg to about 10 mg/kg of total body weight (e.g., about 0.5 μg/kg, about 1 μg/kg, about 50 μg/kg, about 150 μg/kg, about 300 μg/kg, about 750 μg/kg, about 1.5 mg/kg, about 5 mg/kg, or a range defined by any two of the foregoing values), more preferably from about 1 μg/kg to 5 mg/kg of total body weight (e.g., about 3 μg/kg, about 15 μg/kg, about 75 μg/kg, about 300 μg/kg, about 900 μg/kg, about 2 mg/kg, about 4 mg/kg, or a range defined by any two of the foregoing values), and even more preferably from about 0.5 to 15 mg/kg body weight per day (e.g., about 1 mg/kg, about 2.5 mg/kg, about 3 mg/kg, about 6 mg/kg, about 9 mg/kg, about 11 mg/kg, about 13 mg/kg, or a range defined by any two of the foregoing values). Therapeutic or prophylactic efficacy can be monitored by periodic assessment of treated patients. For repeated administrations over several days or longer, depending on the condition, the treatment can be repeated until a desired suppression of disease symptoms occurs. However, other dosage regimens may be useful and are within the scope of the invention. The desired dosage can be delivered by a single bolus administration of the composition, by multiple bolus administrations of the composition, or by continuous infusion administration of the composition.

The composition comprising an effective amount of the inventive immunoglobulin heavy chain polypeptide, the inventive immunoglobulin light chain polypeptide, the inventive DDR1-binding agent, the inventive nucleic acid sequence encoding any of the foregoing, or the inventive vector comprising the inventive nucleic acid sequence can be administered to a mammal using standard administration techniques, including oral, intravenous, intraperitoneal, subcutaneous, pulmonary, transdermal, intramuscular, intranasal, buccal, sublingual, or suppository administration. The composition preferably is suitable for parenteral administration. The term “parenteral,” as used herein, includes intravenous, intramuscular, subcutaneous, rectal, vaginal, and intraperitoneal administration. More preferably, the composition is administered to a mammal using peripheral systemic delivery by intravenous, intraperitoneal, or subcutaneous injection.

Once administered to a mammal (e.g., a human), the biological activity of the inventive DDR1-binding agent can be measured by any suitable method known in the art. For example, the biological activity can be assessed by determining the stability of a particular DDR1-binding agent. In one embodiment of the invention, the DDR1-binding agent (e.g., an antibody) has an in vivo half life between about 30 minutes and 45 days (e.g., about 30 minutes, about 45 minutes, about 1 hour, about 2 hours, about 4 hours, about 6 hours, about 10 hours, about 12 hours, about 1 day, about 5 days, about 10 days, about 15 days, about 25 days, about 35 days, about 40 days, about 45 days, or a range defined by any two of the foregoing values). In another embodiment, the DDR1-binding agent has an in vivo half life between about 2 hours and 20 days (e.g., about 5 hours, about 10 hours, about 15 hours, about 20 hours, about 2 days, about 3 days, about 7 days, about 12 days, about 14 days, about 17 days, about 19 days, or a range defined by any two of the foregoing values). In another embodiment, the DDR1-binding agent has an in vivo half life between about 10 days and about 40 days (e.g., about 10 days, about 13 days, about 16 days, about 18 days, about 20 days, about 23 days, about 26 days, about 29 days, about 30 days, about 33 days, about 37 days, about 38 days, about 39 days, about 40 days, or a range defined by any two of the foregoing values).

The biological activity of a particular DDR1-binding agent also can be assessed by determining its binding affinity to DDR1 or an epitope thereof. The term “affinity” refers to the equilibrium constant for the reversible binding of two agents and is expressed as the dissociation constant (K_{D}). Affinity of a binding agent to a ligand, such as affinity of an antibody for an epitope, can be, for example, from about 1 picomolar (pM) to about 100 micromolar (μM) (e.g., from about 1 picomolar (pM) to about 1 nanomolar (nM), from about 1 nM to about 1 micromolar (μM), or from about 1 μM to about 100 μM). In one embodiment, the DDR1-binding agent can bind to an DDR1 protein with a K_{D} less than or equal to 1 nanomolar (e.g., 0.9 nM, 0.8 nM, 0.7 nM, 0.6 nM, 0.5 nM, 0.4 nM, 0.3 nM, 0.2 nM, 0.1 nM, 0.05 nM, 0.025 nM, 0.01 nM, 0.001 nM, or a range defined by any two of the foregoing values). In another embodiment, the DDR1-binding agent can bind to DDR1 with a K_{D} less than or equal to 200 pM (e.g., 190 pM, 175 pM, 150 pM, 125 pM, 110 pM, 100 pM, 90 pM, 80 pM, 75 pM, 60 pM, 50 pM, 40 pM, 30 pM, 25 pM, 20 pM, 15 pM, 10 pM, 5 pM, 1 pM, or a range defined by any two of the foregoing values). Immunoglobulin affinity for an antigen or epitope of interest can be measured using any art-recognized assay. Such methods include, for example, fluorescence activated cell sorting (FACS), separable beads (e.g., magnetic beads), surface plasmon resonance (SPR), solution phase competition (KINEXA™), antigen panning, and/or ELISA (see, e.g., Janeway et al. (eds.), Immunobiology, 5th ed., Garland Publishing, New York, N.Y., 2001).

The DDR1-binding agent of the invention may be administered alone or in combination with other drugs (e.g., as an adjuvant). For example, the DDR1-binding agent can be administered in combination with other agents for the treatment or prevention of the DDR1-mediated diseases disclosed herein. In this respect, the DDR1-binding agent can be used in combination with at least one other anti-cancer agent including, for example, any chemotherapeutic agent known in the art, ionization radiation, and/or surgery. When the DDR1-mediated disease is fibrosis, the DDR1-binding agent can be used in combination with an anti-inflammatory agent including, for example, a corticosteroid (e.g., prednisone and fluticasone), and/or an immunosuppressive agent, including, for example, methotrexate and cyclosporine. In addition to therapeutic uses, the DDR1-binding agent described herein can be used in diagnostic or research applications. In this respect, the DDR1-binding agent can be used in a method to diagnose a DDR1-mediated disease or disorder. In a similar manner, the DDR1-binding agent can be used in an assay to monitor DDR1 protein levels in a subject being tested for a DDR1-mediated disease or disorder. Research applications include, for example, methods that utilize the DDR1-binding agent and a label to detect a DDR1 protein in a sample, e.g., in a human body fluid or in a cell or tissue extract. The DDR1-binding agent can be used with or without modification, such as covalent or non-covalent labeling with a detectable moiety. For example, the detectable moiety can be a radioisotope (e.g., ³H, ¹⁴C, ³²P, ³⁵S, or ¹²⁵I), a fluorescent or chemiluminescent compound (e.g., fluorescein isothiocyanate, rhodamine, or luciferin), an enzyme (e.g., alkaline phosphatase, beta-galactosidase, or horseradish peroxidase), or prosthetic groups. Any method known in the art for separately conjugating an antigen-binding agent (e.g., an antibody) to a detectable moiety may be employed in the context of the invention (see, e.g., Hunter et al., Nature, 194: 495-496 (1962); David et al., Biochemistry, 13: 1014-1021 (1974); Pain et al., J. Immunol. Meth., 40: 219-230 (1981); and Nygren, J. Histochem. and Cytochem., 30: 407-412 (1982)).

DDR1 protein levels can be measured using the inventive DDR1-binding agent by any suitable method known in the art. Such methods include, for example, radioimmunoassay (RIA), and FACS. Normal or standard expression values of DDR1 can be established using any suitable technique, e.g., by combining a sample comprising, or suspected of comprising, DDR1 with a DDR1-specific antibody under conditions suitable to form an antigen-antibody complex. The antibody is directly or indirectly labeled with a detectable substance to facilitate detection of the bound or unbound antibody. Suitable detectable substances include various enzymes, prosthetic groups, fluorescent materials, luminescent materials, and radioactive materials (see, e.g., Zola, Monoclonal Antibodies: A Manual of Techniques, CRC Press, Inc. (1987)). The amount of DDR1 polypeptide expressed in a sample is then compared with a standard value.

The DDR1-binding agent can be provided in a kit, i.e., a packaged combination of reagents in predetermined amounts with instructions for performing a diagnostic assay. If the DDR1-binding agent is labeled with an enzyme, the kit desirably includes substrates and cofactors required by the enzyme (e.g., a substrate precursor which provides a detectable chromophore or fluorophore). In addition, other additives may be included in the kit, such as stabilizers, buffers (e.g., a blocking buffer or lysis buffer), and the like. The relative amounts of the various reagents can be varied to provide for concentrations in solution of the reagents which substantially optimize the sensitivity of the assay. The reagents may be provided as dry powders (typically lyophilized), including excipients which on dissolution will provide a reagent solution having the appropriate concentration.

The following examples further illustrate the invention but, of course, should not be construed as in any way limiting its scope.

### Example 1

This example demonstrates a method of isolating DDR1-binding agents by screening an antibody library.

An IgG evolvable library based on germline V-gene segments joined to human donor-derived recombined (D)J regions was constructed as described in Bowers et al., Proc. Natl. Acad. Sci. USA, 108(51): 20455-20460 (2011). Nucleic acid sequences encoding IgG heavy chain (HC) and light chain (LC) polypeptides were cloned into separate episomal vectors (Horlick et al., Gene, 243: 187-194 (2000)), with each vector encoding a separate antibiotic selectable marker. The HC vector was formatted such that antibody is both presented on the cell surface as well as secreted into the tissue culture medium, as described in Horlick et al., J. Biol. Chem., 288: 19861-19869 (2013). Vectors encoding HCs and LCs were co-transfected into HEK293 cells, expanded to approximately 10⁹ cells, and then subjected to two rounds each of negative selection against streptavidin (SA)-coupled magnetic beads (Life Technologies, Carlsbad, Calif.) and irrelevant AviTag™ antigen coupled to magnetic beads, followed by a single round of positive selection against SA-coupled magnetic beads coated with biotinylated DDR1. Positively selected cells were diluted and plated in 96-well format at an approximate density of 1-10 cells/well. Resulting colonies were expanded into daughter plates and a portion of each population was tested for binding to DDR1 by FACS array analysis. Supernatants from these plates were also collected to test binding by BIACORE™ and/or ELISA to soluble DDR1. DDR1-binding HC and LC “hits” were then expanded for further characterization.

A number of the isolated cell populations identified from the ABEL library screen were found to bind to DDR1 as measured by FACS. Cells were co-stained with a 4:1 molar ratio of biotinylated DDR1-avi-his (APE01493) to NeutraAvidin (NA)-labeled with DyLight 650 (NA Dyl650) as well as anti-hulgG-PE and analyzed by FACS array. Double positive populations containing cells with high IgG expression that could bind to DDR1 were indicative of potential hits from the library. Plates also were counterscreened with an irrelevant antigen complexed with NA Dyl650 at a 4:1 ratio to identify and eliminate any non-specific binders from the hit pool. Supernatants from antibody expressing cells were screened by both BIACORE™ and ELISA. Over 40 plates were created and screened from eight different sublibraries, each of which contained antibodies based on a different heavy chain gennline V-sequence.

Cells that showed strong specific staining with huDDR1 bio:NA Dyl650 were expanded for sorting and submitted for sequencing to recover the specific HC/LC combinations capable of binding to DDR1. The open reading frames (ORFs) encoding the HCs and LCs of the antibodies found in the cell populations were rescued by PCR. Sequencing revealed that several positive clones were represented by a single HC/LC combination, while other positive clones were represented by multiple HC/LC combinations. Desired HC/LC combinations were identified, and binding activity was confirmed by retransfection of HC and LC vectors into naïve cells. Overall, 15 different HC/LC pairs were confirmed as anti-DDR1-specific hits.

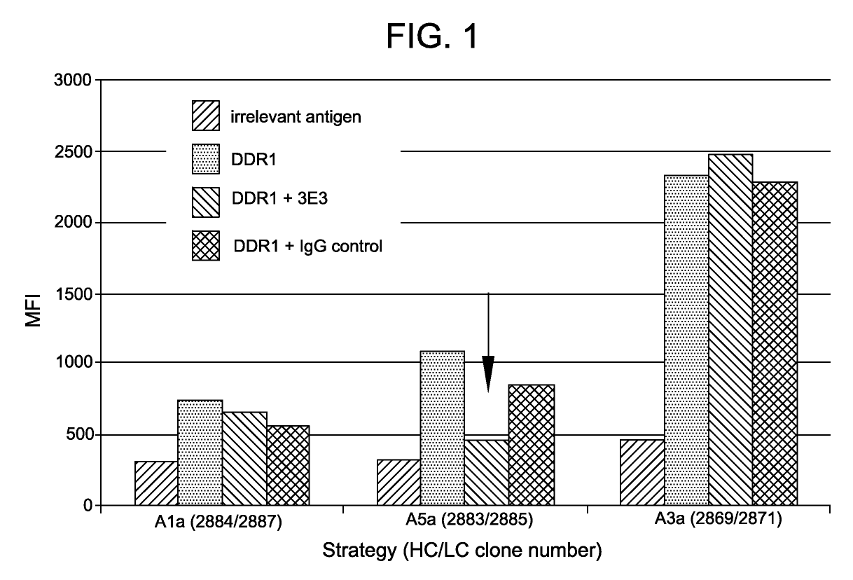

Beyond specific binding to hDDR1 itself, DDR1-specific hits were further characterized by epitope comparison against an inhibitory reference antibody, known as 3E3. This antibody has been reported in the literature as a functional inhibitor that does not interfere with the ability of DDR1 to bind collagen (Carafoli et al., Structure, 20(4): 688-697 (2012)). The V-region sequences for 3E3 were synthesized and the antibody expressed as a fully human IgG1 antibody. Antibody-expressing cells from the ABEL library were incubated either with DDR1:NA Dyl650 complexes alone, or in the presence of 6-8 fold excess 3E3 antibody. The 3E3 antibody exhibited a higher affinity for DDR1 as compared to select early library hits, as shown in FIG. 1.

Two deletion mutations of DDR1 were generated as an alternative approach to map antibodies that bind to or near the collagen binding site on DDR1. The first DDR1 mutation, designated APE01929 and also referred to as DDR1^{Δcol}#1, contained a deletion of residues 48-60, corresponding to loop 1 on the DDR1 protein. The second deletion mutation, designated APE01930 and also referred to as DDR1^{Δcol}#2, contained a deletion of residues 105-112, corresponding to loop 3 on the DDR1 protein. Similar deletions have been shown to abolish signaling as measured by receptor phosphorylation (Abdulhussein et al., J. Biol. Chem., 279(30): 31462-31470 (2004)). Antibodies identified from certain ABEL library strategies could bind to full length DDR1, but not the DDR1 deletion mutants, suggesting that these antibodies may map to the deleted regions.

Antibodies also were characterized for their ability to bind to DDR1 orthologs from the cynomolgus monkey (Macaca fascicularis) (cynoDDR1) and mouse (muDDR1) (R&D Systems, Minneapolis, Minn.), and human DDR2 (huDDR2). Purified antibody from all of the ABEL library strategies bound to two isoforms of cynoDDR1. Overall, the strategies fell into four distinct bins, (1) a 3E3 competitive monoclonal antibody (A5), (2) monoclonal antibodies that failed to bind to the DDR1^{Δcol} mutations (A1, A3, A4, and A11), (3) a monoclonal antibody that does not bind to mouse (A7), and (4) a monoclonal antibody that binds to mouse but did not compete with either the DDR1^{Δcol} mutations or the 3E3 reference antibody (A7). None of the antibodies demonstrated detectable binding to human DDR-2, indicating the specificity of the antibodies for DDR1. A summary of the antibody characterization for the ABEL library strategies is set forth in Table 1.

TABLE 1

| | 3E3 | Binds to | Binds to | Binds to |
|---|---|---|---|---|
| Strategy | Competition | DDR1^{Δcol} | muDDR1 | DDR2 |
| A1a | − | − | + | − |
| A3a | − | − | + | − |
| A4a | − | − | + | − |
| A11a | − | − | + | − |
| A5a | + | + | + | − |
| A7a | − | + | − | − |

The results of this example demonstrate a method of isolating DDR1-specific antibodies by screening an antibody library.

### Example 2

This example describes affinity maturation of the DDR1-specific monoclonal antibodies identified in Example 1.

Stable cell lines co-expressing the nucleic acid sequences encoding the HC and LC of each antibody described in Example 1 were transfected with an expression vector encoding activation-induced cytidine deaminase (AID) to initiate somatic hypermutation (SHM). A total of 17 different cell lines based on eight distinct antibody strategies were established and used for affinity maturation. Cell populations were selected based on both IgG expression and binding to antigen, collected by flow cytometry as a bulk population, and then expanded for further analysis. This process was repeated iteratively to accumulate SHM-derived mutations in the variable regions of both the heavy and light chains and their derivatives for each strategy. As affinity of each antibody improved, the stringency of selection was increased. For example, the complexed biotinylated DDR1-bio was replaced by DDR1 that was directly labeled with DyL650 in order to select under monomeric (i.e., non-avid) binding conditions, concentrations of labeled DDR-1 were progressively reduced, and washing stringency was progressively increased. Enrichment of emerging populations representing antibodies with potential improving mutations were observed between several rounds.

To identify mutations of interest for further manipulation, HC and LC DNA sequences were obtained from the FACS sorted cell populations using two different methods: (1) standard Sanger sequencing and (2) next generation sequencing (NGS, also referred to as deep sequencing analysis) using methodology provided by Illumina, Inc. (San Diego, Calif.). For both procedures, HC and LC ORFs were rescued by PCR from episomal vectors isolated from FACS-sorted cells. For Sanger sequencing, approximately 50 sequences were examined per FACS round to identify potential improving mutations. Once mutations were identified, they were recombined in the HC and LC DNAs, produced as purified antibody, and tested for improvements in binding affinity.

Additional enriched HC and LC mutations were identified from deep sequence analyses that ranged from approximately 30,000 to 100,000 DNA template reads per round of antigen-based selection on sorted cells. The additional sequencing data from NGS allowed for identification of an increased number of potential improving mutations as well as adding statistical confidence for mutations selected to be reincorporated and tested in the full HC/LC contexts. Samples were submitted for NGS post round zero (RO) as a baseline and for each subsequent round after round 2 (R2).

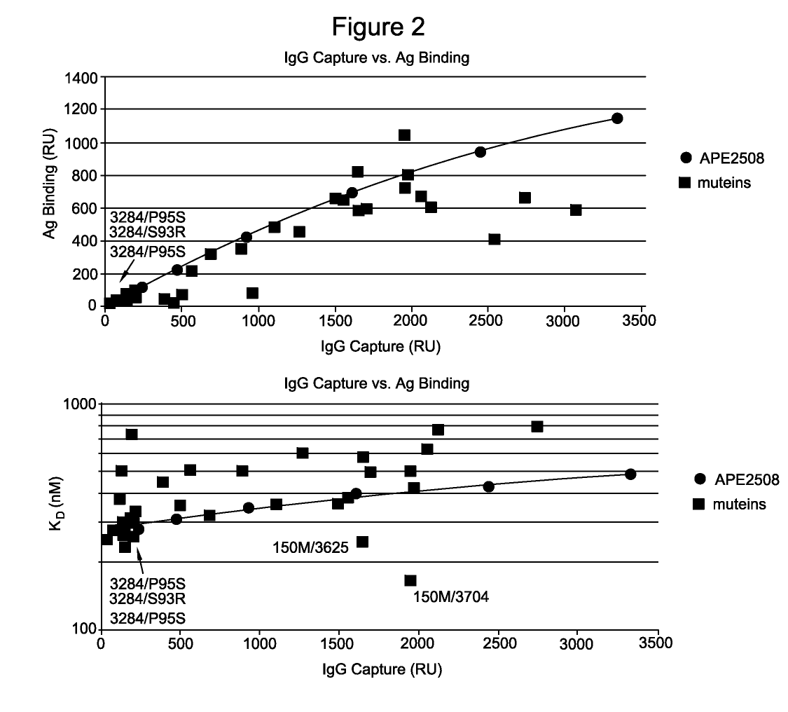

Large numbers of SHM events were tested by BIACORE™ using supernatants harvested from a transfection array paired with the best HC or LC identified at the time. A control antibody containing the HC or LC context without incorporated mutations was also spiked in at varying concentrations to provide a reference curve to help identify improving or non-productive mutations. Both total antigen captured as well as KD values were used to assess improvement contributions from individual mutations. These were plotted against total antibody captured and graphed, as shown in FIG. 2. SHM events with a confirmed impact on binding were recombined for further analysis.

To determine binding kinetics, monomeric huDDR1 was immobilized onto the surface of a chip and antibody flowed over at a range of concentrations. BIACORE™ analysis was carried out by immobilizing DDR1 fused to six histidine residues at the C-terminus (DDR1-his) at low density directly onto a BIACORE™ chip surface. Antibodies were flowed at a range of concentrations over the chip to determine kinetic constants using BIACORE™ evaluation software.

Following characterization of antibodies encoding various recombinations of mutations, the final set of antibodies was selected. The SHM events and designations of the HC and LC clones are set forth in Table 2, and the binding kinetics of the tested antibodies are set forth in Table 3.

TABLE 2

| Antibody | | |
|---|---|---|
| Designation | HC SHM events | LC SHM events |
| APE02829 | V2, S31N, I50M, A71N, F100Y | S29R, ins34YLA, S94L, G50D, S93R |
| APE02832 | V2L, S31N, I50M, A71N, T30S, F100Y | S29R, ins34YLA, S94L, G50D, S93R |
| APE02834 | V2L, S31N, I50M, A71N, S82N, F100Y | S29R, ins34YLA, S94L, G50D, S93R |
| APE02835 | V2L, S31N, I50M, A71N, S82N, D101N | S29R, ins34YLA, S94L, G50D, S93R |
| APE02837 | V2L, S31N, I50M, A71N, T30S, S82N, F100Y | S29R, ins34YLA, S94L, G50D, S93R |
| APE02839 | V2L, S31N, I50M, A71N, T30S, F100Y, D101N | S29R, ins34YLA, S94L, G50D, S93R |
| APE02840 | V2L, S31N, I50M, A71N, T30S, S82N, F100Y, D101N | S29R, ins34YLA, S94L, G50D, S93R |

TABLE 3

| Antibody Designation | kₐ (M⁻¹sec⁻¹) | kd (sec⁻¹) | KD (nM) |
|---|---|---|---|
| APE02832 | 1.80E+06 | 3.30E−04 | 0.2 |
| APE02835 | 1.86E+06 | 4.4E−04 | 0.2 |
| APE02839 | 1.46E+06 | 5.50E−04 | 0.4 |
| APE02840 | 1.72E+06 | 4.70E−04 | 0.3 |

The monoclonal antibodies described in Table 3 were tested for the ability to recognize two different isoforms of cynoDDR1, mouse (mu) DDR1 (R&D Systems, Minneapolis, Minn.), and huDDR2 (R&D Systems, Minneapolis, Minn.; cat#2538-DR-040). All antibodies were able to recognize both isoforms of cynoDDR1 as well as muDDR1. None of the antibodies were found to cross-react with huDDR2. Cross-reactivity to cynoDDR1 as compared to huDDR1 was measured in an ELISA assay, and the antibodies exhibited similar affinities for cynoDDR1 and huDDR1.

The results of this example demonstrate a method of generating the inventive immunoglobulin heavy and light chain polypeptides, which exhibit high affinity for DDR1.

### Example 3

This example demonstrates the activity of the inventive immunoglobulin heavy and light chain polypeptides in vitro.

All of the antibodies described in Table 2 of Example 2 were tested for the ability to bind to cell surface DDR1. Each of the antibodies bound to DDR1 expressed on the following cell lines: T47D, which is a breast cancer cell line expressing endogenous DDR1, 293 cells overexpressing DDR1 (293/DDR1, Gilead, Foster City, Calif.), and U2OS cells overexpressing a DDR1 fusion protein (DiscoveRx, Fremont, Calif.). The antibodies did not bind to 293 cells overexpressing DDR2 (293/DDR2), nor did they bind to untransfected 293 cells.

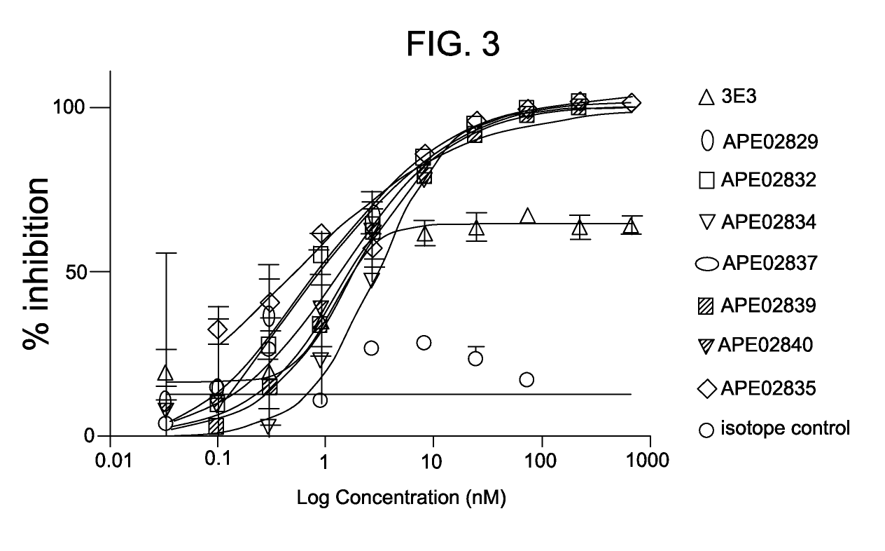

The antibodies described in Table 3 also were analyzed for the ability to inhibit collagen-mediated phosphorylation of DDR1 in a U2OS cell-based pathway indicator assay (DiscoveRx, Fremont, Calif.). This U2OS cell line was engineered to express both a DDR1 receptor fused with a partial and non-functional β-galactosidase (β-gal) enzyme fragment. When the receptor is phosphorylated after ligand stimulation, forced complementation with an SH2 domain fusion to a complementary n-gal fragment occurs to create a complete β-gal enzyme. Chemiluminescent substrate is then added for detection. The results of this assay are set forth in FIG. 3, which indicate that the antibodies are functional antagonists in this assay.

The results of this example demonstrate that the inventive immunoglobulin heavy and light chain polypeptides can antagonize DDR1 signaling.

### Example 4

This example demonstrates the activity of the inventive immunoglobulin heavy and light chain polypeptides in vitro.

Additional DDR-1-binding heavy chain polypeptides were identified by screening the ABEL library described in Example 1 and were affinity matured as described in Example 2. These HC polypeptides comprise the amino acids sequences of SEQ ID NO: 26, SEQ ID NO: 27, SEQ ID NO: 28, and SEQ ID NO: 29. Each of these HC sequences were paired with a light chain polypeptide comprising SEQ ID NO: 22, and the resulting antibodies were named as set forth in Table 4.

TABLE 4

| Antibody | HC SEQ ID | LC SEQ ID |
|---|---|---|
| Designation | NO: | NO: |
| APE05551 | 26 | 22 |
| APE05552 | 27 | 22 |
| APE05570 | 28 | 22 |
| APE05571 | 29 | 22 |

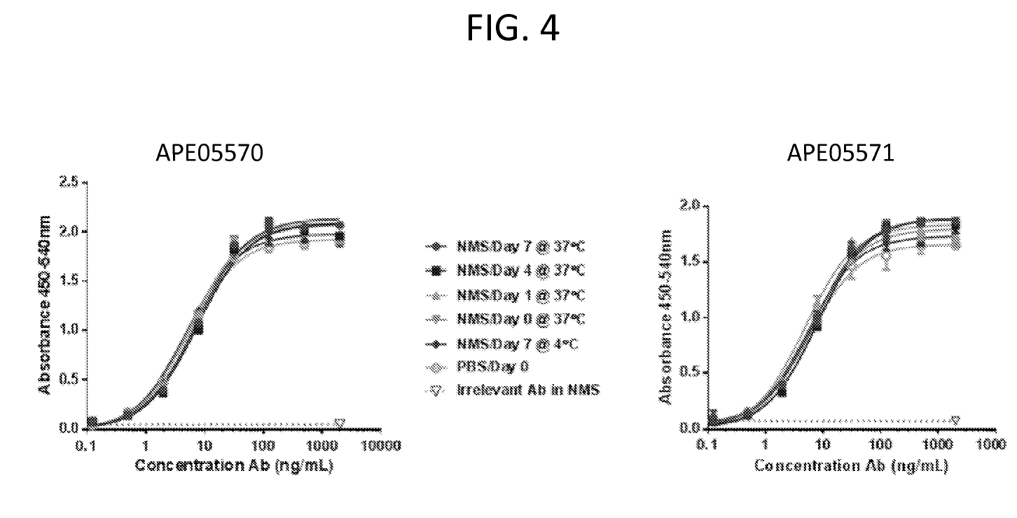

The APE05570 and APE05571 antibodies described in Table 4 were tested for the ability to bind cell surface DDR in mouse serum. The antibodies were incubated in normal mouse serum for 1, 4, or 7 days at 37° C. Varying concentrations of the antibodies were then analyzed for ability to bind to DDR1 by an ELISA assay. Briefly, hDDR1-avi-his was coated on plates at 1 μg/m overnight at 4° C. and washed three times in phosphate buffered saline with Tween (PBST). Plates were then blocked with 3% BSA/PBS (200 μl/well) for one hour. The antibodies were then diluted, added to the washed plates, and incubated for two hours at room temperature. Bound antibodies were detected with goat anti HuIgG-(H+L chain)-horseradish peroxidase (HRP) at a dilution of 1:10,000. After washing the plates, 100 μl of 3,3′,5,5′-tetramethylbenzidine (TMB) substrate was added to detect antibody, and plates were read at 450 nm using a SpectraMax™ microplate reader (Molecular Devices, LLC., Sunnyvale, Calif.). Both the APE05570 and APE05571 antibodies stably bound DDR1 in mouse serum during the studied time period, as shown in FIG. 4.

The antibodies described in Table 4 and the APE02840 antibody described in Example 3 were analyzed for their ability to inhibit collagen-mediated phophorylation of DDR1 in T47D cells. T47D cells endogenously expressing DDR1 were plated at 25,000 cells/well. 24 hours later, each of the monoclonal antibodies was preincubated at a dose titration and incubated on the cells for 30 minutes. The antibody 3E3 (also designated APE1555) is a reference antibody, which is a partial inhibitor of collagen-mediated phosphorylation of DDR1 and was tested as a control. Collagen I (BD Biosciences, San Jose, Calif.) was then added to a final concentration of 40 μg/ml to stimulate the DDR1 receptor. Plates were incubated overnight for 16 hours+/−30 minutes before cells were lysed. Cell lysates were prepared for analysis using the Phospho-DDR1 ELISA kit (Cell Signaling Technology, Inc., Danvers, Mass.). Data was analyzed using GraphPad Prism software (GraphPad, San Diego, Calif.) using a log (inhibitor) vs. response (three parameter) curve fit. IC50 was calculated based on the midpoint of the dose response curve. The results of this assay are set forth in Table 5, and demonstrate that the antibodies are functional DDR1 antagonists in this assay.

TABLE 5

| | 3E3 ref | APE05551 | APE05552 | APE05570 | APE05571 | APE02840 |
|---|---|---|---|---|---|---|
| P-DDR1 IC50 (nM) | 0.031* | 26.12 | 27.58 | 24.96 | 19.33 | 13.42 |
| Tₘ (° C.) | n/d | 66.7 | 65.2 | 72.3 | 71.4 | [0061] |
| (thermofluor) | | | | | | |

Surface plasmon resonance (SPR) analyses were carried out using a BIACORE™ T200 system (GE Healthcare, Little Chalfont, Buckinghamshire, UK) by immobilizing DDR1 fused to six histidine residues at the C-terminus (DDR1-his) at low density directly onto a BIACORE™ chip surface. Antibodies were flowed at a range of concentrations over the chip to determine kinetic constants using BIACORE™ evaluation software. Experimental parameters were chosen to ensure that saturation was reached at the highest antigen concentrations and Rₘₐₓ values were kept under 70 resonance units (RU), which is a measure of surface plamon resonance signal.

anti-Human IgG (Fc-specific, ≈10,000 RU) (GE Healthcare, Little Chalfont, Buckinghamshire, UK) was immobilized on a BIACORE™CMS chip using EDC-activated amine coupling chemistry. Antibodies (0.5 μg/mL, 50 second capture time at 10 μl/minute) were then captured using this surface to a target capture level of ˜125 RU. Monomeric soluble human DDR1-avi-his was then flowed over captured antibody (300 second association, 600 second dissociation) using a three-fold serial dilution series from 200 nM to 0.27 nM. Runs were performed at 25° C. and HBS-EP+, pH 7.6 buffer was used for all dilutions (Teknova, Hollister, Calif.). Captured antibody and antigen were removed between each cycle using two regeneration steps with 3M MgCl₂ (60 seconds contact time) to ensure a fresh binding surface for each concentration of antigen. The resulting sensorgrams were fit globally using a 1:1 binding model to calculate on- and off-rates (ka and kd, respectively) and dissociation constants as a measure of overall affinity (KD). The results of this experiment are set forth in Table 6, and demonstrate that the tested antibodies exhibited high affinity for huDDR1.

TABLE 6

| | | | | | Chi² | Ligand | Rmax |
|---|---|---|---|---|---|---|---|
| Antibody | Antigen | KD (M) | ka (1/Ms) | kd (1/s) | (RU²) | Level (RU) | (RU) |
| APE05551.2 | APE1469.6 | 5.14E−08 | 5.51E+05 | 2.84E−02 | 0.797 | 110.5 | 65.1 |
| | (hDDR1) | | | | | | |
| APE05552.2 | APE1469.6 | 4.38E−08 | 5.07E+05 | 2.22E−02 | 0.171 | 129.9 | 76.6 |
| | (hDDR1) | | | | | | |
| APE05570.1 | APE1469.6 | 3.51E−08 | 7.37E+05 | 2.59E−02 | 0.76 | 116.5 | 64.8 |
| | (hDDR1) | | | | | | |
| APE05571.1 | APE1469.6 | 4.70E−08 | 4.96E+05 | 2.33E−02 | 0.684 | 121.6 | 72.1 |
| | (hDDR1) | | | | | | |
| APE02840.03 | APE1469.6 | 3.17E−08 | 6.14E+05 | 1.95E−02 | 0.201 | 113 | 67.5 |
| | (hDDR1) | | | | | | |

The results of this example demonstrate that the inventive immunoglobulin heavy and light chain polypeptides, which exhibit high affinity for DDR1 and antagonize DDR1 signaling.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An isolated immunoglobulin heavy chain or light chain polypeptide, wherein:
(I) the immunoglobulin heavy chain polypeptide comprises the amino acid sequence of SEQ ID NO: 1, wherein optionally one or more of residues 2, 30, 31, 51, 78, 85, 102, 107, and 108 of SEQ ID NO: 1 is replaced with a different amino acid residue;
and
(II) the immunoglobulin light chain polypeptide comprises
(a) the amino acid sequence of SEQ ID NO: 13, wherein optionally residue 30 of SEQ ID NO: 13 is replaced with a different amino acid residue;
or
(b) the amino acid sequence of SEQ ID NO: 15, wherein optionally (i) one or more of residues 37, 58, 101, and 102 of SEQ ID NO: 15 is replaced with a different amino acid residue, and/or (ii) an amino acid sequence comprising YLA is inserted into SEQ ID NO: 15 after residue 42;
or
(c) the amino acid sequence of SEQ ID NO: 23, wherein optionally reside 25 and/or residue 66 of SEQ ID NO: 23 is/are replaced with a different amino acid residue.

2. (canceled)

3. The isolated immunoglobulin heavy chain polypeptide of claim 1, wherein the immunoglobulin heavy chain polypeptide comprises SEQ ID NO: 1 in which:
(a) residue 2 of SEQ ID NO: 1 is replaced with a leucine (L) residue,
(b) residue 30 of SEQ ID NO: 1 is replaced with a serine (S) residue,
(c) residue 31 of SEQ ID NO: 1 is replaced with an asparagine (N) residue,
(d) residue 51 of SEQ ID NO: 1 is replaced with a methionine (M) residue,
(e) residue 78 of SEQ ID NO: 1 is replaced with an asparagine (N) residue,
(f) residue 85 of SEQ ID NO: 1 is replaced with an asparagine (N) residue,
(g) residue 102 of SEQ ID NO: 1 is replaced with a threonine (T) residue,
(h) residue 107 of SEQ ID NO: 1 is replaced with a tyrosine (Y) residue,
(i) residue 108 of SEQ ID NO: 1 is replaced with an asparagine (N) residue, or
(j) any combination of (a)-(h).

4. The isolated immunoglobulin heavy chain polypeptide of claim 3, which comprises an amino acid sequence of any one of SEQ ID NOs: 2-12 and SEQ ID NOs: 26-29.

5.-8. (canceled)

9. The isolated immunoglobulin light chain polypeptide of claim 1, wherein the immunoglobulin light chain polypeptide comprises SEQ ID NO: 13 in which residue 30 of SEQ ID NO: 13 is replaced with an isoleucine (I) residue.

10. The isolated immunoglobulin light chain polypeptide of claim 9, which comprises the amino acid sequence of SEQ ID NO: 14.

11.-12. (canceled)

13. The isolated immunoglobulin light chain polypeptide of claim 1, wherein the immunoglobulin light chain polypeptide comprises SEQ ID NO: 1 in which
(a) residue 37 of SEQ ID NO: 15 is replaced with an arginine (R) residue,
(b) residue 58 of SEQ ID NO: 15 is replaced with an aspartic acid (D) residue,
(c) residue 101 of SEQ ID NO: 15 is replaced with an arginine (R) residue,
(d) residue 102 of SEQ ID NO: 15 is replaced with a leucine (L) residue, or
(e) any combination of (a)-(d).

14. The isolated immunoglobulin light chain polypeptide of claim 13, which comprises the amino acid sequence of SEQ ID NO: 16.

15. The isolated immunoglobulin light chain polypeptide of claim 1, wherein the immunoglobulin light chain polypeptide comprises SEQ ID NO: 15 in which an amino acid sequence comprising YLA is inserted into SEQ ID NO: 15 after residue 42.

16. The isolated immunoglobulin light chain polypeptide of claim 15, which comprises the amino acid sequence of any one of SEQ ID NOs: 17-22.

17. (canceled)

18. The isolated immunoglobulin light chain polypeptide of claim 1, wherein the immunoglobulin light chain polypeptide comprises SEQ ID NO: 23 in which residue 25 and/or residue 66 of SEQ ID NO: 23 is/are replaced with a different amino acid.

19. The isolated immunoglobulin light chain polypeptide of claim 18, wherein residue 25 of SEQ ID NO: 23 is replaced with a threonine (T) residue and/or residue 66 of SEQ ID NO: 23 is replaced with a glutamic acid (E) residue.

20. The isolated immunoglobulin light chain polypeptide of claim 19, which comprises the amino acid sequence of SEQ ID NO: 24 or SEQ ID NO: 25.

21. An isolated immunoglobulin light chain polypeptide which comprises an amino acid sequence that is at least 90% identical to any one of SEQ ID NO: 13-25, or an isolated immunoglobulin light chain polypeptide which comprises an amino acid sequence that is at least 90% identical to any one of SEQ ID NOs: 26-29; or which binds to the same epitope of Discoidin Domain Receptor Family, Member 1 (DDR1) as the immunoglobulin light chain polypeptide or immunoglobulin heavy chain polypeptide of claim 1.

22.-25. (canceled)

26. An isolated Discoidin Domain Receptor Family, Member 1 (DDR1)-binding agent comprising (a) the immunoglobulin heavy chain polypeptide claim 1, or one, two, or three complementarity determining regions (CDRs) thereof; and/or (b) the immunoglobulin light chain polypeptide of claim 1, or one, two, or three CDRs thereof.

27. The isolated DDR1-binding agent of claim 26, which is an antibody, an antibody conjugate, or an antigen-binding fragment thereof.

28. The isolated DDR1-binding agent of claim 26, which is an antibody fragment selected from F(ab′)₂, Fab′, Fab, Fv, scFv, dsFv, dAb, and a single chain binding polypeptide.

29.-36. (canceled)

37. An isolated or purified nucleic acid sequence encoding the isolated immunoglobulin heavy chain polypeptide or isolated immunoglobulin light chain polypeptide of claim for a the DDR1-binding agent comprising same, optionally in a vector or isolated cell.

38.-39. (canceled)

40. A composition comprising the isolated DDR1-binding agent of claim 26 and a pharmaceutically acceptable carrier.

41. A method of treating a DDR1-mediated disorder in a mammal, which method comprises administering an effective amount of the composition of claim 40 to a mammal having DDR1-mediated disorder, whereupon the DDR1-mediated disorder is treated in the mammal; wherein the disorder is optionally cancer or fibrosis.

42. The method of claim 41, wherein the DDR1-mediated disorder is lung cancer, breast cancer, ovarian cancer, renal fibrosis, or lung fibrosis.

43.-45. (canceled)

46. The method of claim 41, wherein (a) the half-life of the DDR1-binding agent in the mammal is between 30 minutes and 45 days; (b) the DDR1-binding agent binds to DDR1 with a K_{D} between about 1 picomolar (pM) and about 100 micromolar (μM); or both (a) and (b).

47. (canceled)
